(19) United States
(12) Patent Application Publication
(10) Pub. No.: US 2024/0368230 A1
(43) Pub. Date: Nov. 7, 2024

(54) CELL-PENETRATING PEPTIDES COMPRISING MULTIPLE 5-MER ALPHA-HELIX FRAGMENTS AND USES THEREOF

(71) Applicant: ArtProBio Co., Ltd., Seoul (KR)

(72) Inventors: Yong-Beom LIM, Seoul (KR); Dong in KIM, Seoul (KR)

(73) Assignee: ArtProBio Co., Ltd., Seoul (KR)

(21) Appl. No.: 18/393,158

(22) Filed: Dec. 21, 2023

(30) Foreign Application Priority Data
May 2, 2023 (KR) 10-2023-0057293

(51) Int. Cl. C07K 14/16; A61K 47/64

(52) U.S. Cl. CPC C07K 14/16; A61K 47/64

(57) ABSTRACT

The present disclosure relates to a novel cell-penetrating peptide having a stabilized α-helical structure without having a negative charge. Since it has no cytotoxicity, has 4 times or higher cell-penetrating ability than the Tat cell-penetrating peptide and has ear-specific targeting ability due to its sequence and self-stabilized α-helical structure, it can be usefully used for various therapeutic drug delivery systems and drug therapy technologies.

### CROSS-REFERENCE TO RELATED APPLICATION

This application claims priority to Korean Patent Application No. 10-2023-0057293 filed on May 2, 2023, and all the benefits accruing therefrom under 35 U.S.C. § 119, the contents of which in its entirety are herein incorporated by reference.

### SEQUENCE LISTING

The instant application contains a Sequence Listing which has been submitted electronically in a computer readable Sequence Listing XML format and is hereby incorporated by reference in its entirety. Said computer readable Sequence Listing in XML format was created on Dec. 21, 2023, is named G1035-26601_SequenceListing.xml and is 45,678 bytes in size.

### BACKGROUND

### 1. Field

The present disclosure relates to a cell-penetrating peptide containing multiple arginines and containing one or more specific 5-mer alpha-helix fragment, and a use thereof.

### 2. Description of the Related Art

Although peptides, proteins, nucleic acids, etc. have great potential as therapeutic substances, their use is very limited because they cannot penetrate the cell membrane of target cells. In addition, even small-molecule compounds often cannot penetrate the lipid bilayer of the cell membrane due to their specific properties or structures. For this reason, there have been attempts to deliver the therapeutic substances into cells through electroporation, membrane fusion using liposomes, heat shock, etc. However, it is difficult to deliver the substances into cells without damaging the cell membrane while maintaining the activity of the substances.

In this situation, cell-penetrating proteins or peptides have been attracting attentions recently. Among them, the most researched one is TAT protein derived from human immunodeficiency virus 1 (HIV-1). It is known that, in the TAT protein consisting of 86 amino acids, a peptide consisting of amino acids 47-57 has the cell-penetrating function. As similar examples, it is known that amino acids 267-300 of the VP22 protein of HSV-1, amino acids 339-355 of Drosophila antennapedia (Antp), artificially synthesized positively charged peptides function as cell-penetrating peptides. Based on these facts, researches are being conducted to combine the cell-penetrating peptides with cargo substances such as proteins or nucleic acids for delivery into cells.

Meanwhile, cell membrane-penetrating peptides composed of hydrophobic peptides were also developed. A representative example is MTS (membrane-translocating sequence). MTS is composed of 16 amino acids derived from human FGF-4 protein and is mainly composed of hydrophobic amino acids (sequence: AAVALLPAVLLALLAP). The hydrophobic peptide is known to penetrate the hydrophobic region inside the lipid bilayer constituting the cell membrane and deliver a substance directly to the cytoplasm. Besides, cell-penetrating peptides are being developed from various proteins of various biological species and cell membrane-penetrating peptides have also been developed by designing artificial sequences.

Accordingly, it is very important to secure a candidate with excellent cell-penetrating ability than previously known cell-penetrating peptides and secure technological superiority for its application.

The inventors of the present disclosure have explored a novel cell-penetrating peptide (CPP) with a new secondary structure and completed the present disclosure by confirming that it can overcome the problems of existing delivery materials.

### REFERENCES OF THE RELATED ART

### Patent Documents

Patent document 1. Korean Patent Publication No. 10-2014-0128971.

### SUMMARY

The present disclosure is directed to providing a cell-penetrating peptide with superior in-vivo drug delivery and stability properties.

The present disclosure is also directed to providing a use of the novel cell-penetrating peptide.

The present disclosure provides a cell-penetrating peptide containing a repeating unit represented by SEQ ID NO 1.

| [SEQ ID NO 1] | |
|---|---|
| | Xaa1-Xaa2-Xaa3-Xaa4-Xaa5 |

In SEQ ID NO 1, each of Xaa1 to Xaa5 is independently any one selected from a group consisting of Gln (Q), Ala (A), Arg (R) and Asn (N), the repeating unit being repeated 2-10 times.

One or more amino acid selected from SEQ ID NO 1 is an L- or D-amino acid.

The repeating unit may be repeated 2-6 times.

The repeating unit may be repeated 3-5 times.

In SEQ ID NO 1, each of Xaa1 and Xaa5 may independently be Gln (Q) or Asn (N) and each of Xaa2 to Xaa4 may independently be Ala (A) or Arg (R).

At least one of Xaa2 to Xaa4 may be Ala (A).

The repeating unit may be any one selected from sequences represented by SEQ ID NOS 5-10.

The cell-penetrating peptide may further contain a linker represented by any one of SEQ ID NOS 2-4 between the N-terminal and the C-terminal and the repeating unit.

| [SEQ ID NO 2] | |
|---|---|
| | Xaa6 |
| [SEQ ID NO 3] | |
| | Xaa7-Xaa8 |
| [SEQ ID NO 4] | |
| | Xaa9-Xaa10-Xaa11 |

In SEQ ID NOS 2-4, is independently an α,α-disubstituted amino acid selected from a group consisting of Ala (A), Arg (R), Ile (I), Leu (L), Met (M), Val (V), Gly (G) and 2-aminoisobutyric acid (Aib, U), and one or more amino acid selected from SEQ ID NOS 2-4 is an L- or D-amino acid.

Each of Xaa6 to Xaa11 may independently be an amino acid selected from Ala (A), Arg (R) and 2-aminoisobutyric acid (Aib, U).

The cell-penetrating peptide may be any one selected from sequences represented by SEQ ID NOS 11-33.

The cell-penetrating peptide may be any one selected from sequences represented by SEQ ID NOS 20-29 and 33.

The cell-penetrating peptide may be any one selected from a group consisting of SEQ ID NOS 20, 21, 22, 23, 24 and 33.

The cell-penetrating peptide may be a cell-penetrating carrier peptide.

The cell-penetrating peptide may be accumulated in the nucleus of a cell.

The cell-penetrating peptide may be a cell-penetrating carrier peptide targeting the ear.

The present disclosure is also directed to providing an intracellular delivery system including the cell-penetrating peptide.

A cargo to be delivered intracellularly may be bound to the terminal of the cell-penetrating peptide.

The intracellular delivery system may target the ear.

The present disclosure is also directed to providing composition for intracellular delivery of an active ingredient, which contains the cell-penetrating peptide for delivery of an active ingredient into a cell by the cell-penetrating peptide.

The composition may target the ear.

The composition may deliver an active ingredient topically into the nucleus of a cell.

The present disclosure relates to a novel cell-penetrating peptide having a stabilized α-helical structure without having a negative charge. Since it has very low cytotoxicity, has 4 times or higher cell-penetrating ability than the Tat cell-penetrating peptide and has ear-specific targeting ability due to its sequence and α-helical structure, it can be usefully used for various therapeutic drug delivery systems and drug therapy technologies.

### BRIEF DESCRIPTION OF THE DRAWINGS

FIG. 1 schematically shows the domain structure of Rev, wherein OLIGO represents an oligomerization domain, NES represents a nuclear export signal, NTD represents an N-terminal domain and CTD represents a C-terminal domain.

FIG. 2 schematically shows the binding mechanism between the Rev protein and RRE RNA. The insert in FIG. 2 shows the structure of Rev NTD (PDB 2X7L) after binding of the Rev protein to the IIB region of RRE RNA.

FIG. 3 schematically shows the sequence of an oligomerization-defective cRev NTD (Rev₉₋₆₅) mutant. The Leu residue at position 12, the Val residue at position 16, the Leu residue at position 18, the Ile residue at position 55 and the Leu residue at position 60 in cRev are substituted with Ala, and the corresponding regions in the sequence are marked with purple color. The sequence shown with orange color is the sequence added for intein-mediated cyclization.

FIG. 4 schematically shows the intein-mediated cyclization of the Rev protein expressed in bacteria.

FIGS. 5-7 show analysis results for the cRev and /Rev proteins. FIG. 5 shows the analysis result for the proteins purified by FPLC, FIG. 6 shows the SDS-PAGE analysis result, and FIG. 7 shows the MALDI-TOF MS analysis result of cRev.

FIG. 8 shows an SEC (size-exclusion chromatography) analysis result for carbonic anhydrase, FIG. 9 shows an SEC (size-exclusion chromatography) analysis result for cytochrome C, FIG. 10 shows an SEC (size-exclusion chromatography) analysis result for aprotinin, FIG. 11 shows an SEC (size-exclusion chromatography) analysis result for cRev NTD (Rev₉₋₆₅), and FIG. 12 shows an SEC (size-exclusion chromatography) analysis result for /Rev NTD (Rev₉₋₆₅).

FIG. 13 shows the CD spectrum of /Rev NTD (Rev₉₋₆₅) (/Rev) and FIG. 14 shows the CD spectrum of cRev NTD (Rev₉₋₆₅) (cRev).

FIG. 15 shows the CD spectrum of a cRev NTD (Rev₉₋₆₅) (cRev)-IIB RNA complex.

FIG. 16 shows the UV melting curves of IIB RNA and a cRev-IIB complex.

FIG. 17 shows the fluorescence anisotropy analysis result for a mixture of cRev NTD (Rev₉₋₆₅) (cRev) and Cy3-IIB RNA.

FIG. 18 shows the EMSA (electrophoretic mobility shift assay) result for a mixture of cRev NTD (Rev₉₋₆₅) (cRev) and wild-type IIB (RRE IIB WT) or mutant IIB (RRE IIB MT).

FIG. 19 shows the EMSA (electrophoretic mobility shift assay) result for a mixture of cRev NTD (Rev₉₋₆₅) (cRev) or /Rev NTD (Rev₉₋₆₅) (/Rev) and wild-type IIB (RRE IIB WT).

FIGS. 20A to 20F show the NMR solution structure of cRev NTD (Rev₉₋₆₅) (cRev) and /Rev NTD (Rev₉₋₆₅) (/Rev) at 298 K. FIG. 20A shows a set of 20 conformers exhibiting the solution structure of /Rev NTD (Rev₉₋₆₅) (/Rev) (PDB 8X3P) and FIG. 20B shows a set of 20 conformers exhibiting the solution structure of cRev NTD (Rev₉₋₆₅) (cRev) (PDB 8X30). FIG. 20C shows the monomer structure of /Rev NTD (Rev₉₋₆₅) (/Rev) and FIG. 20D shows the monomer structure of cRev NTD (Rev₉₋₆₅) (cRev). FIG. 20E schematically compares the secondary structure elements of /Rev NTD (Rev₉₋₆₅) (/Rev), cRev NTD (Rev₉₋₆₅) (cRev) and the Rev protein in a Rev-IIB complex. FIG. 20F shows the sequence analysis result of an alpha-helix fragment in the Rev protein.

FIG. 21 shows the ¹H/¹⁵N HSQC spectrum of cRev.

FIG. 22 shows the ¹H/¹⁵N HSQC spectrum of /Rev.

FIG. 23 shows a result of analyzing the chemical shift index related to secondary structure using the NMR data of cRev and/Rev.

FIGS. 24A to 24C show a Rev-RRE recognition model which schematically shows how the intrinsic disorder of Rev affects multimerization and specificity. FIG. 24A shows the energy landscape of a Rev conformer (Eₐ₁>Eₐ₂), FIG. 24B shows the initial process of a Rev-RRE recognition model, and FIG. 24C shows a result of analyzing the raison d'etre of the intrinsic disorder of Rev.

FIG. 25 shows the sequences of cell-penetrating peptides represented by SEQ ID NOS 15-24 (CPP5 to CPP13 and CPP11d). The lowercase letters stand for D-amino acids and U stands for Aib (2-aminoisobutyric acid).

FIG. 26 shows the MALDI-TOF MS spectra of cell-penetrating peptides prepared in Examples 1-14 (CPP1 to CPP13 and CPP11d).

FIG. 27A to 27C shows the CD spectra of 6 cell-penetrating peptides prepared in Examples 1-4 and 6-7.

FIG. 28 and FIG. 29 show the CD spectra of 9 cell-penetrating peptides prepared in Examples 5-6 and 8-14.

FIG. 30 schematically shows the hydrogen bonding of a cell-penetrating peptide prepared in Example 11 (CPP11).

FIG. 31 shows the CD spectra of a cell-penetrating peptide prepared in Example 11 (CPP11) at different temperature conditions (4° C., 20° C., 36° C., 52° C., 68° C. and 84° C.).

FIG. 32 shows the CD spectrum ([θ]₂₂₂/[θ]₂₀₈) of a cell-penetrating peptide prepared in Example 11 (CPP11) depending on concentration.

FIG. 33 shows the CD spectra of the disordered Tat peptide (Comparative Example) derived from HIV-1 and a cell-penetrating peptide prepared in Example 11 (CPP11).

FIG. 34 shows the flow cytometry result for Hela cells treated with a cell-penetrating peptide prepared in Example 10 (CPP10), a cell-penetrating peptide prepared in Example 11 (CPP11) or an existing cell-penetrating peptide (Tat) (n=3) (mean±S.D.).

FIG. 35 shows the flow cytometry result for Hela cells, HCT116 cells and 293T cells treated with a cell-penetrating peptide prepared in Example 10 (CPP10), a cell-penetrating peptide prepared in Example 11 (CPP11) or an existing cell-penetrating peptide (Tat) (n=3) (mean±S.D.).

FIG. 36 shows a result of treating Hela cells with a cell-penetrating peptide prepared in Example 10 (CPP10), a cell-penetrating peptide prepared in Example 11 (CPP11) or a cell-penetrating peptide prepared in Example 14 (CPP11d) and measuring cytotoxicity with a WST-8 reagent.

FIG. 37 shows the flow cytometry result for Hela cells treated with a cell-penetrating peptide prepared in Example 11 (CPP11) at different concentrations.

FIG. 38 shows a result of treating Hela cells with a cell-penetrating peptide prepared in Example 10 (CPP10), a cell-penetrating peptide prepared in Example 11 (CPP11) or the Tat peptide in various conditions to probe the cellular uptake pathway by flow cytometry (n=3) (mean±S.D.).

FIG. 39 shows the CLSM (confocal laser scanning microscopy) images of HeLa cells treated with a cell-penetrating peptide prepared in Example 11 (CPP11) or the Tat peptide. The yellow lines indicate cell nuclei stained with DAPI.

FIGS. 40A and 40B show the CLSM (confocal laser scanning microscopy) images of HeLa cells treated with a cell-penetrating peptide prepared in Example 11 (CPP11) or the Tat peptide, comparing cellular localization behavior more specifically. The yellow lines indicate cell nuclei stained with DAPI.

FIG. 41 shows the EMSA (electrophoretic mobility shift assay) images of yeast tRNA for a cell-penetrating peptide prepared in Example 11 (CPP11) or the Tat peptide.

FIG. 42 shows a result of quantifying R_{f} (retention factor) from the EMSA result of FIG. 41.

FIGS. 43A and 43B show a result of analyzing the nonspecific electrostatic interaction between a cell-penetrating peptide prepared in Example 11 (CPP11) or the Tat peptide and heterologous RNA (IIB RNA). FIG. 43A shows the EMSA analysis result for the cell-penetrating peptide prepared in Example 11 (CPP11) or the Tat peptide, and FIG. 43B shows a result of quantifying R from FIG. 43A.

FIG. 44A shows the in-vivo fluorescence images of mice treated with a cell-penetrating peptide of Example 10 (CPP10). FIG. 44B shows the in-vivo fluorescence image of mice to which the cell-penetrating peptide of Example 10 (CPP10) was administered by IP (intraperitoneal) injection and IV injection. FIG. 44C shows the in-vivo fluorescence images of mice to which the cell-penetrating peptide of Example 10 (CPP10), a cell-penetrating peptide of Example 11 (CPP11) or a cell-penetrating peptide of Example 14 (CPP11d) was administered by IV injection.

FIG. 45 shows the IVIS image of major organs extracted 4 hours after the injection of a cell-penetrating peptide prepared in Example 11 (CPP11) via the tail vein.

FIGS. 46A and 46B show the IVIS images of the outside (FIG. 46A) and inside (FIG. 46B) of the head (including the ear) 4 hours after the injection of a cell-penetrating peptide prepared in Example 11 (CPP11) or a buffer solution (PBS; w/o treatment) via the tail vein.

FIG. 47 shows the IVIS images of the cochlea (inner ear) separated from the head and the ear 4 hours after the injection of a cell-penetrating peptide prepared in Example 11 (CPP11) via the tail vein.

FIG. 48 shows the IVIS images of the cochlea (inner ear) separated from the ear of an experimental animal after administration of a cell-penetrating peptide prepared in Example 11 (CPP11) or treatment with a buffer solution (PBS; w/o treatment).

FIGS. 49A-49E show the CD spectra of five cell-penetrating peptides (CPP14 to 18) prepared in Examples 15-19 (CPP14 to 18).

FIG. 50 shows the IVIS image of living experimental animals after the administration of cell-penetrating peptides of Examples 15-19 (CPP14 to 18) by IV injection.

FIG. 51 shows the IVIS image of living experimental animals after the administration of a cell-penetrating peptide of Example 11 (CPP11) and cell-penetrating peptides of Example 15, 16 and 18 (CPP14, 15 and 17) by IV injection.

FIGS. 52A-52C show the CD spectra of cell-penetrating peptides prepared in Examples 20-22 (CPP19 to 21).

FIG. 53 shows the IVIS image of living experimental animals after administration of cell-penetrating peptides of Examples 20-22 (CPP19 to 21) (‘3’, ‘2’ and ‘1’ in order) by IV injection.

FIG. 54 shows the IVIS image of living experimental animals after administration of an existing cell-penetrating peptides (Rev ARM, Tat, penetratin and R8) or a cell-penetrating peptide of Example 11 (CPP11) (′1′, ‘2’, ‘3’, ‘4’ and ‘5’ in order) by IV injection.

FIG. 55 shows the IVIS image obtained by removing background signals from the IVIS image for the existing cell-penetrating peptides (Rev ARM, Tat, penetratin and R8) or the cell-penetrating peptide of Example 11 (CPP11) (′1′, ‘2’, ‘3’, ‘4’ and ‘5’ in order) (FIG. 54).

FIG. 56 shows the amino acid sequence of a fusion protein wherein CPP22 is fused with the ZsGreen protein (CPP22-ZsGreen).

FIG. 57 shows the flow cytometry result for Hela cells treated only with the ZsGreen protein at various concentrations (10, 20 or 50 μM).

FIG. 58 shows the flow cytometry result for Hela cells treated with a CPP22-ZsGreen fusion protein at various concentrations (10, 20 or 50 μM).

FIG. 59 shows a result of analyzing Hela cells treated with the ZsGreen protein (50 μM) or a CPP22-ZsGreen fusion protein (50 μM) by confocal microscopy.

### DETAILED DESCRIPTION

Hereinafter, the present disclosure is described in detail.

In an aspect, the present disclosure relates to a cell-penetrating peptide containing a repeating unit represented by SEQ ID NO 1.

| [SEQ ID NO 1] | |
|---|---|
| | Xaa1-Xaa2-Xaa3-Xaa4-Xaa5 |

In SEQ ID NO 1, each of Xaa1 to Xaa5 is independently any one selected from a group consisting of Gln (Q), Ala (A), Arg (R) and Asn (N), the repeating unit being repeated 2-10 times.

The one or more amino acid selected from SEQ ID NO 1 is an L- or D-amino acid.

In the present specification, the term “peptide” or “polypeptide” refers to a linear molecule formed by linkage of amino acid residues by peptide bonds. It is composed of 4-70, specifically 4-50, more specifically 10-50, most specifically 20-50, amino acid residues.

The cell-penetrating peptide represented by SEQ ID NO 1 is one designed to contain a disordered sequence (IDR/IDP) (QARRN) of the arginine-rich motif (ARM) domain present in the NTD peptide in the Rev protein of HIV (human immunodeficiency virus) and a disordered sequence expected therefrom as repeating units. The inventors of the present disclosure have confirmed through experiments that a peptide containing the sequences has superior cell membrane-penetrating activity and have discovered through this that it can be used as a drug delivery system capable of delivering substances into cells.

The cell-penetrating peptide of SEQ ID NO 5 of the present disclosure is a fragment derived from the Rev protein of HIV (human immunodeficiency virus), which is a retrovirus, as a repeating sequence. The present disclosure is advantageous in that the novel cell-penetrating peptides are designed by combining the 5-mer repeating unit sequence in tandem repeats in the presence or absence of appropriate spacers and the designed cell-penetrating peptides have unique structural and functional characteristics.

Specifically, the Rev protein of HIV-1 (human immunodeficiency virus type 1) is a regulatory protein that is essential in self-replication. This protein is believed to induce the expression of structural proteins by facilitating the export of structural gene mRNAs from the nucleus to the cytoplasm. The Rev protein exerts its function by binding directly to the Rev-responsive element (RRE) which is a cis-acting element existing in the env gene of HIV-I RNA. The RRE is a complex RNA secondary structure consisting of a central stem (I′), a small stem (I) and five stem-loops (II, III, IV, V and VI). The inventors of the present disclosure have investigated which region in the Rev regulatory protein is essential for the reaction with RRE RNA. As a result, it was revealed that the Rev NTD is essential and, particularly, the secondary structure is essential for interaction with the RRE RNA. The NTD of the Rev protein contains an arginine-rich motif (ARM) domain has a transient (unstable) α-helix structure. More specifically, it contains an amino acid sequence derived from the Rev protein of human immunodeficiency virus type 1 (hereinafter, also referred to as HIV-1) having a transient α-helix structure. The inventors of the present disclosure transformed the transient α-helix structure into a highly stable α-helix structure in the designed peptides by combining the 5-mer repeating unit sequence in tandem repeats in the presence or absence of appropriate spacers. In doing so, the newly designed peptides not only have highly stable α-helix structures but also have cell-penetrating and ear-targeting functions (namely, cell-penetrating perfect α-helix; CPPα).

In a specific exemplary embodiment of the present disclosure, since the cell-penetrating peptide of the present disclosure does not aggregate with each other and exits in the form of a monomer in solution, there is an advantage that the efficiency of cellular uptake/penetration is not decreased even if the concentration increases.

In a specific exemplary embodiment of the present disclosure, the repeating unit represented by SEQ ID NO 1 may be repeated one or more times. For example, it may be repeated 2 or more, 2-50, 2-10, 2-6, 2-5 or 3-5 times. In terms of the effect of intracellular delivery and accumulation, the repeating unit represented by SEQ ID NO 1 may be repeated 3-5 times.

In SEQ ID NO 1, each of Xaa1 and Xaa5 may independently be Gln (Q) or Asn (N), and each of Xaa2 to Xaa4 may independently be Ala (A) or Arg (R).

In the cell-penetrating peptide of the present disclosure, at least one of Xaa2 to Xaa4 may be Ala (A).

In another specific exemplary embodiment of the present disclosure, the repeating unit represented by SEQ ID NO 1 is composed of five amino acids (5-mer), and the amino acid Gln (Q) or Asn (N) at positions i and i+4 forms hydrogen bonding to make the α-helical structure more rigid.

In another specific exemplary embodiment of the present disclosure, in the repeating unit represented by SEQ ID NO 1, one or more selected from Xaa1 to Xaa5 may be a D-amino acid residue. When the D-amino acid residue is used, the accumulation efficiency at the target site is increased significantly.

Specifically, the repeating unit represented by SEQ ID NO 1 may be any one selected from sequences represented by SEQ ID NOS 5-10. It may be specifically selected from SEQ IDS NO 5-7, more specifically selected from SEQ ID NO 5 or 6, most specifically SEQ ID NO 5.

In an exemplary embodiment of the present disclosure, the cell-penetrating peptide may further contain a linker, and the linker may be located at one or more of: (i) the N-terminal of the cell-penetrating peptide; (ii) the C-terminal of the cell-penetrating peptide; and (iii) in between the repeating unit represented by SEQ ID NO 1.

The linker may be represented by any one of SEQ ID NOS 2-4.

| [SEQ ID NO 2] | |
|---|---|
| | Xaa6 |
| [SEQ ID NO 3] | |
| | Xaa7-Xaa8 |
| [SEQ ID NO 4] | |
| | Xaa9-Xaa10-Xaa11 |

In SEQ ID NOS 2-4,
each of Xaa6 to Xaa11 is independently an α,α-disubstituted amino acid selected from a group consisting of Ala (A), Arg (R), Ile (I), Leu (L), Met (M), Val (V), Gly (G) and 2-aminoisobutyric acid (Aib, U). One or more amino acid selected from SEQ ID NOS 2-4 is an L- or D-amino acid.

In an exemplary embodiment of the present disclosure, a structure represented by Chemical Formula 1 may be formed when the cell-penetrating peptide further contains a linker.

A(BA)ₙ [Chemical Formula 1]

In Chemical Formula 1, n may be an integer of 1 or greater, specifically an integer greater than 1, e.g., one or more selected from an integer of 1-10, an integer of 2-9, an integer of 2-8, an integer of 2-7, an integer of 2-5 and an integer of 3-5.

In the chemical formula, “A” represents a linker (any one selected from SEQ ID NOS 2-4) and “B” represents a linker repeating unit represented by SEQ ID NO 1.

The segment A and the segment B are connected in a substantially linear fashion as opposed to a branched or star-shaped fashion.

Accordingly, the cell-penetrating peptide according to the present disclosure may be represented by the following general formulas.

Xaa6-(Xaa1-Xaa2-Xaa3-Xaa4-Xaa5-Xaa6)ₙ [General Formula 1]

Xaa7-Xaa8-(Xaa1-Xaa2-Xaa3-Xaa4-Xaa5-Xaa7-Xaa8)ₙ [General Formula 2]

Xaa9-Xaa10-Xaa11-(Xaa1-Xaa2-Xaa3-Xaa4-Xaa5-Xaa9-Xaa10-Xaa11)ₙ [General Formula 3]

In General Formulas 1-3, n may be an integer of 1 or greater, specifically an integer greater than 1, e.g., one or more selected from an integer of 1-10, an integer of 2-9, an integer of 2-8, an integer of 2-7, an integer of 2-5 and an integer of 3-5.

In the general formulas, Xaa1 to Xaa11 are the same as described above.

The cell-penetrating peptide of the present disclosure can be interpreted also to include an amino acid sequence exhibiting substantial identity to the amino acid sequence described above.

In the present disclosure, the substantial identity may mean a sequence exhibiting a homology of at least 80%, at least 90% or at least 95% when aligned to match as much as possible with another sequence and analyzed using an algorithm commonly used in the art.

The cell-penetrating peptide may include conservative substitution and/or modification of one or more amino acid.

In the present disclosure, the modification may include deletion or addition of amino acids that has minimal effect on the properties and secondary structure of the cell-penetrating peptide of the present disclosure. For example, the cell-penetrating peptide may be conjugated to a signal (or leader) sequence at the N-terminal of a protein involved in protein transfer either co-translationally or post-translationally. In addition, the cell-penetrating peptide may be conjugated to another sequence or spacer for identification, purification or synthesis of the cell-penetrating peptide.

In the present disclosure, the conservative substitution means the replacement of an amino acid with another amino acid having similar structural and/or chemical properties. The amino acid substitution can generally occur based on the similarity in polarity, charge, solubility, hydrophobicity, hydrophilicity and/or amphipathic nature of the residue.

The cell-penetrating peptide of the present disclosure may have variants and the variants may be named as follows.

In the present disclosure, a specific position in an amino acid sequence may refer to an amino acid present or substituted at that position. An amino acid at a specific position may be expressed variously. For example, “position 003” may also be expressed as “position 3”, “amino acid 3” or “3rd amino acid”. In addition, when an amino acid located at the 3rd position is arginine (R), it may be expressed as “R3” or “Arg3”.

The amino acid substitution can be expressed by describing the amino acid before the substitution, the position and the amino acid to be substituted in order. The amino acid can be expressed using the conventional one-letter and three-letter codes. For example, when serine, which is an amino acid at position 5 of a specific sequence, is substituted with cysteine, it may be described as “S5C” or “Ser5Cys”.

Any amino acid at a specific position may be referred to as “X” or “Xaa”. For example, X6 refers to any amino acid at position 6. In addition, when the amino acid to be substituted is expressed as X, it means that it is substituted with an amino acid different from the amino acid present before substitution. For example, “V6X” indicates that V at position 6 is substituted with any amino acid other than V.

Different alterations can be expressed by simultaneously describing several types of amino acids using symbols, such as “/” or “,”. For example, when the amino acid (F) at position 20 is substituted with S or C, it may be described as F20S/C or F20S,C. As another example, F/S20C means that the amino acid F or S at position 20 before substitution is substituted with C.

Multiple mutations can be described using “+”. For example, “R3C+T36C” mean that arginine, which is an amino acid at position 3, is substituted with cysteine, and threonine, which is an amino acid at position 36, is substituted with cysteine, respectively.

In a specific exemplary embodiment of the present disclosure, the cell-penetrating peptide may be a polypeptide containing the ARM domain of the Rev protein of a retrovirus. The cell-penetrating peptide may contain sequences represented by SEQ ID NOS 11-33, specifically any one selected from SEQ ID NOS 15-33, more specifically any one selected from SEQ ID NOS 20-33, further more specifically any one selected from SEQ ID NOS 20-29 and 33, even more specifically any one selected from SEQ ID NOS 20, 21, 22, 23, 24 and 33, most specifically SEQ ID NO 21 or 24.

The cell-penetrating peptide is accumulated in the nucleolus of a cell as a cell-penetrating carrier peptide. When administered in vivo, the cell-penetrating carrier peptide targets the ear.

In another aspect, the present disclosure provides an intracellular delivery system including the cell-penetrating peptide.

A cargo to be delivered intracellularly may be bound to the terminal of the cell-penetrating peptide.

In the present specification, the term “intracellular delivery system” refers to a delivery system that can penetrate the cell membrane and access the surface of the nucleolus. The intracellular delivery system may target the ear.

In the present specification, the term “cargo” refers to a chemical substance, small molecule, nucleic acid, etc. that can be bound to a peptide acting as a cell-penetrating peptide and transported into a cell.

The substance that can be bound to the cell-penetrating peptide according to the present disclosure, i.e., the substance that can be a cargo, is various. For example, it includes a protein (polypeptide), a nucleic acid (polynucleotide), a chemical substance (drug, contrast agent, etc.), etc., although not being limited thereto.

For example, it can be a drug, a contrast agent (e.g., a T1 contrast agent, a T2 contrast agent such as a superparamagnetic substance, a radioisotope, etc.), a fluorescent marker, a dyeing agent, etc., although not being limited thereto. The polypeptide is a polymer of amino acids composed of two or more residues and includes a peptide and a protein. The polypeptide may be, for example, proteins that are involved in cell immortalization (e.g., SV40 large T antigen and telomerase), anti-apoptotic proteins (e.g., mutant p53 and BclxL), antibodies, cancer genes (e.g., ras, myc, HPV E6/E7 and adenoviridae Ela), cell cycle regulating proteins (e.g., cyclin and cyclin-dependent phosphorylase) or enzymes (e.g., green fluorescent protein, beta-galactosidase and chloramphenicol acetyltransferase), although not being limited thereto. Also, the nucleic acid may be, for example, RNA, DNA or cDNA, and the sequence of the nucleic acid may be an encoding site sequence or a non-coding site sequence (e.g., an antisense oligonucleotide or a siRNA). Nucleotides as nucleic acid cargos may be standard nucleotides (e.g., adenosine, cytosine, guanine, thymine, inosine and uracil) or analogs (e.g., phosphorothioate nucleotides). For example, the nucleic acid cargo may be an antisense sequence consisting of a phosphorothioate nucleotide or RNAi.

In a specific exemplary embodiment of the present disclosure, the cell-penetrating peptide according to the present disclosure or a substance bound thereto penetrates the cell membrane with very high efficiency and is accumulated and remains in the cytoplasm, on the surface of the nucleus, and within the nucleus.

In another aspect, the present disclosure relates to a composition for intracellular delivery of an active ingredient, which contains the cell-penetrating peptide for delivery of an active ingredient into a cell by the cell-penetrating peptide.

The composition may target the ear and the composition may topically deliver the active ingredient to the nucleolus within a cell.

The active ingredient of the composition has the same meaning as the ‘cargo’ mentioned above with regard to the intracellular delivery system and refers to a chemical substance, small molecule, polypeptide, nucleic acid, etc. that can be bound to a peptide acting as a cell-penetrating peptide and delivered into a cell. For more specific examples, refer to the description of the cargo mentioned above.

In another aspect, the present disclosure provides a method for delivering cargo to be delivered into a cell, which includes a step of contacting an intracellular delivery system wherein a cargo to be delivered into a cell is bound to the terminal of the cell-penetrating peptide with a cell.

The description of the intracellular delivery system used in the present disclosure will be omitted to avoid unnecessary redundancy.

When the cell-penetrating peptide with the cargo bound is given the opportunity to contact the cell membrane in vitro or in vivo, the cargo bound to the peptide is transported into the cell. There is no special condition required for the contact between the intracellular delivery system and the cell membrane, e.g., limitation in time, temperature, concentration, etc., and the contact can be carried out under general conditions for penetration of the cell membrane known in the art.

The intracellular delivery system may be prepared by adding one or more pharmaceutically acceptable carrier in addition to the active ingredient described above. The pharmaceutically acceptable carrier may be saline, sterile water, Ringer's solution, buffered saline, dextrose solution, maltodextrin solution, glycerol, ethanol, liposome or a mixture thereof, and other common additives such as an antioxidant, a buffer, a bacteriostat, etc. may be added if necessary. In addition, a diluent, a dispersant, a surfactant, a binder or a lubricant may be added additionally to prepare an injectable formulation such as an aqueous solution, a suspension, an emulsion, etc., a pill, a capsule, a granule or a tablet. In addition, a target organ-specific antibody or other ligands may be bound to the carrier for specific action on the target organ. Furthermore, the formulation may be prepared depending on diseases or ingredients using an appropriate method known in the art or the methods described in Remington's Pharmaceutical Science (recent edition), Mack Publishing Company, Easton PA.

The intracellular delivery system may be delivered by injection via intravenous, intraperitoneal, intramuscular, subcutaneous, intradermal, nasal, mucosal, inhalational or oral routes. The administration dosage may be varied depending on the body weight, age, sex, health condition and diet of a subject, administration time, administration method, excretion rate, the severity of a disease, etc. A daily administration dosage is about 0.01-100 mg/kg, specifically 1-50 mg/kg and it may be more specifically administered once or several times a day.

The SEQ ID NOS of the present specification are summarized in the following table.

TABLE 1

| | Sequence (N → C) | SEQ ID NO |
|---|---|---|
| SEQ ID NO 1 | Xaa1-Xaa2-Xaa3-Xaa4-Xaa5 | SEQ ID NO 1 |
| | The sequence may be repeated 2-10 | |
| | times, X may independently be any | |
| | one selected from Gln (Q), Ala (A), | |
| | Arg (R), Asn (N), D-Gln (Q), D-Ala | |
| | (A), D-Arg (R) or D-Asn (N). | |
| SEQ ID NO 2 | Xaa6 | SEQ ID NO 2 |
| | X may be any one selected from Ala | |
| | (A), Arg (R), Ile (I), Leu (L), Met | |
| | (M), Val (V), D-Ala (A), D-Arg (R), | |
| | D-Ile (I), D-Leu (L), D- Met (M), | |
| | D-Val (V) and 2-aminoisobutyric | |
| | acid (Aib, U). | |
| SEQ ID NO 3 | Xaa7-Xaa8 | SEQ ID NO 3 |
| | X may independently be any one | |
| | selected from Ala (A), Arg (R), Ile | |
| | (I), Leu (L), Met (M), Val(V), D- | |
| | Ala (A), D-Arg (R), D-Ile (I), D- | |
| | Leu (L), D-Met (M), D-Val (V) and | |
| | 2-aminoisobutyric acid (Aib, U). | |
| SEQ ID NO 4 | Xaa9-Xaa10-Xaa11 | SEQ ID NO 4 |
| | X may independently be any one | |
| | selected from Ala (A), Arg (R), Ile | |
| | (I), Leu (L), Met (M), Val(V), D- | |
| | Ala (A), D-Arg (R), D-Ile (I), D- | |
| | Leu (L), D-Met (M), D-Val (V) and | |
| | 2-amino-isobutyric acid (Aib, U). | |
| α-Helix | QARRN | SEQ ID NO 5 |
| fragment | | |
| α-Helix | QRARN | SEQ ID NO 6 |
| fragment | | |
| α-Helix | QRRAN | SEQ ID NO 7 |
| fragment | | |
| α-Helix | NRRAQ | SEQ ID NO 8 |
| fragment | | |
| α-Helix | NRARQ | SEQ ID NO 9 |
| fragment | | |
| α-Helix | NARRQ | SEQ ID NO 10 |
| fragment | | |
| Example 1 | RQARRNRQARRNRQARRNRQARRN | SEQ ID NO 11 |
| (CPP1) | | |
| Example 2 | RQARRNRQARRNRQARRNRQARRNRQARRN | SEQ ID NO 12 |
| (CPP2) | | |
| Example 3 | RQARRNRQARRNRQARRNRQARRNRQARRNRQARR | SEQ ID NO 13 |
| (CPP3) | N | |
| Example 4 | AUAUARQARRNRQARRNRQARRNAUAUA | SEQ ID NO 14 |
| (CPP4) | | |
| Example 5 | UAURQARRNUAURQARRNUAURQARRNUAURQARR | SEQ ID NO 15 |
| (CPP5) | NUAU | |
| Example 6 | QARRNQARRNQARRNQARRN | SEQ ID NO 16 |
| (CPP6) | | |
| Example 7 | QARRNQARRNQARRNQARRNQARRN | SEQ ID NO 17 |
| (CPP7) | | |
| Example 8 | UQARRNUQARRNUQARRNUQARRNU | SEQ ID NO 18 |
| (CPP8) | | |
| Example 9 | UAQARRNUAQARRNUAQARRNUAQARRNUA | SEQ ID NO 19 |
| (CPP9) | | |
| Example 10 | UAUQARRNUAUQARRNUAUQARRNUAU | SEQ ID NO 20 |
| (CPP10) | | |
| Example 11 | UAUQARRNUAUQARRNUAUQARRNUAUQARRNUAU | SEQ ID NO 21 |
| (CPP11) | | |
| Example 12 | AAAQARRNAAAQARRNAAAQARRNAAAQARRNAAA | SEQ ID NO 22 |
| (CPP12) | | |
| Example 13 | UAUQARRNUAUQARRNUAUQARRNUAUQARRNUAU | SEQ ID NO 23 |
| (CPP13) | QARRNUAU | |
| Example 14 | UaUgarrnUaUqarrnUaUqarrnUaUqarrnUaU | SEQ ID NO 24 |
| (CPP11d) | The lower-case letters indicate | |
| | D-amino acids. | |
| Example 15 | UAUQRARNUAUQRARNUAUQRARNUAUQRARNUAU | SEQ ID NO 25 |
| (CPP14) | | |
| Example 16 | UAUQRRANUAUQRRANUAUQRRANUAUQRRANUAU | SEQ ID NO 26 |
| (CPP15) | | |
| Example 17 | UAUNRRAQUAUNRRAQUAUNRRAQUAUNRRAQUAU | SEQ ID NO 27 |
| (CPP16) | | |
| Example 18 | UAUNRARQUAUNRARQUAUNRARQUAUNRARQUAU | SEQ ID NO 28 |
| (CPP17) | | |
| Example 19 | UAUNARRQUAUNARRQUAUNARRQUAUNARRQUAU | SEQ ID NO 29 |
| (CPP18) | | |
| Example 20 | UAUQARRNUAUQARRNUAUQARRNUGUQARRNUAU | SEQ ID NO 30 |
| (CPP19) | | |
| Example 21 | UAUQARRNUAUQARRNUGUQARRNUGUQARRNUAU | SEQ ID NO 31 |
| (CPP20) | | |
| Example 22 | UAUQARRNUGUQARRNUGUQARRNUGUQARRNUAU | SEQ ID NO 32 |
| (CPP21) | | |
| Example 23 | AAAQARRNAAAQARRNAAAQARRNAAAQARRNAAA | SEQ ID NO 33 |
| (CPP22) | QARRNAAA | |
| Example 24 | GSHMASAAAQARRNAAAQARRNAAAQARRNAAAQA | SEQ ID NO 34 |
| CPP22- | RRNAAAQARRNAAAGSGSGSGSGPVATMAQSKHGL | |
| ZsGreen | TKEMTMKYRMEGCVDGHKFVITGEGIGYPFKGKQA | |
| | INLCVVEGGPLPFAEDILSAAFMYGNRVFTEYPQD | |
| | IGSHMASAAAQARRNAAAQARRNAAAQARRNAAAQ | |
| | ARRNAAAQARRNAAAGSGSGSGSGPVATMAQSKHG | |
| | LTKEMTMKYRMEGCVDGHKFVITGEGIGYPFKGKQ | |
| | ADYFKNSCPAGYTWDRSFLFEDGAVCICNADITVS | |
| | VEENCMYHESKFYGVNFPADGPVMKKMTDNWEPSC | |
| | EKIIPVPKQGILKGDVSMYLLLKDGGRLRCQFDTV | |
| | YKAKSVPRKMPDWHFIQHKLTREDRSDAKNQKWHL | |
| | TEHAIASGSALP | |
| ZsGreen | MAQSKHGLTKEMTMKYRMEGCVDGHKFVITGEGIG | SEQ ID NO 35 |
| | YPFKGKQAINLCVVEGGPLPFAEDILSAAFMYGNR | |
| | VFTEYPQDIADYFKNSCPAGYTWDRSFLFEDGAVC | |
| | ICNADITVSVEENCMYHESKFYGVNFPADGPVMKK | |
| | MTDNWEPSCEKIIPVPKQGILKGDVSMYLLLKDGG | |
| | RLRCQFDTVYKAKSVPRKMPDWHFIQHKLTREDRS | |
| | DAKNQKWHLTEHAIASGSALP | |
| /Rev | DEDALKAARAIKFLYQSNPPPNPEGTRQARRNRRR | SEQ ID NO 36 |
| | RWRERQRQIHSISERILSTLG | |
| Rev wild | CRGGDEDALKAARAIKFLYQSNPPPNPEGTRQARR | SEQ ID NO 37 |
| type | NRRRRWRERQRQIHSISERILSTLGGMRM | |
| cRev | CRGGDEDALKAARAIKFLYQSNPPPNPEGTRQARR | SEQ ID NO 38 |
| | NRRRRWRERQRQIHSASERIASTLGGMRM | |
| Wild-type | GGACCUGGUAUGGGCGCAGCGCAAGCUGACGGUAC | SEQ ID NO 39 |
| IIB or RRE | AGGCCAGGUCC | |
| IIB WT | | |
| IIB mutant: | GGACCUGGUAUCGGCGCAGCGCAAGCUGACGGUAG | SEQ ID NO 40 |
| | AGGCCAGGUCC | |
| Tat peptide | Tyr-Gly-Arg-Lys-Lys-Arg-Arg-Gln-Arg- | SEQ ID NO 41 |
| | Arg-Arg | |
| Rev_ARM | TRQARRNRRRRWRERQR | SEQ ID NO 42 |
| Penetratin | RQIKIWFQNRRMKWKK | SEQ ID NO 43 |
| R8 | RRRRRRRR | SEQ ID NO 44 |

Hereinafter, the present disclosure is described more specifically through specific examples. However, the examples are only for describing the present disclosure more specifically and it will be obvious to those having ordinary knowledge in the art that the scope of the present disclosure is not limited by them.

### <Experimental Materials>

General chemical substances were purchased from Thermo Fisher Scientific (USA) and Samchun (Korea). Fmoc-protected amino acids (D- or L-) and coupling reagents were purchased from Novabiochem (Germany), Anaspec (USA) and AAPPtec (USA). Rink amide MBHA resin LL was purchased from Novabiochem (Germany).

HOBt (1-hydroxybenzotriazole) and HATU (1-[bis(dimethylamino)methylene]-1H-1,2,3-triazolo[4,5-b]pyridinium 3-oxide hexafluorophosphate) were purchased from Anaspec and AAPPTec, respectively. gBlocks gene fragments were purchased from Integrated DNA Technologies (USA). Competent E. coli cells were purchased from Novagen (USA). ¹⁵NH₄Cl and ¹³C-D-glucose were acquired from Cambridge Isotope Laboratories (USA).

### Experimental Methods

### CD Spectroscopy

The concentration of peptides/proteins was 5-20 μM in general. Spectra were obtained using a Jasco J-1500 CD spectrometer (JASCO, Japan) or a Chirascan CD spectrometer (Applied Photophysics, UK). Scanning was carried out using a cuvette with a path length of 1 mm. MRE (mean residue ellipticity) was calculated per amino acid residue.

### UV Melting Analysis

UV melting experiments for free RNA (2 μM) and a protein-RNA complex (1:1 molar ratio) were conducted using an Agilent 8453 diode array UV-Vis spectrophotometer equipped with a Peltier temperature controller. Spectra were measured at 260 nm using a cuvette with a path length of 1 cm under the heating rate condition of 2° C./min. The measurement was repeated 3 times and the average was taken. Prior to the measurement, the RNA sample was heated at 90° C. for 2 minutes and then annealed on ice. The melting temperature (Tₘ) was calculated with the Origin software (OriginLab, USA) using a two-state or multi-state model.

### EMSA (Electrophoretic Mobility Shift Assay)

Wild-type IIB and mutant IIB were purchased from Integrated DNA technologies.

<Sequences of wild-type IIB and mutant IIB>
Wild-type IIB:
SEQ ID NO 39
5′-GGACCUGGUAUGGGCGCAGCGCAAGCUGACGGUACAGGCCAGGUCC-
IIB mutant:
SEQ ID NO 40
5′-GGACCUGGUAUCGGCGCAGCGCAAGCUGACGGUAGAGGCCAGGUCC-

### (1) EMSA of Wild-Type IIB and Mutant IIB

A peptide (sample) and ³²P-labeled RNA were mixed with a binding buffer (10 mM HEPES (pH 7.5), 100 mM KCl, 1 mM MgCl₂, 0.5 mM EDTA, 10% glycerol and 50 mg/mL yeast tRNA). After serially diluting the peptide with the binding buffer, RNA (10 pM) of the same volume was added. The resulting mixture was incubated at room temperature for 30 minutes and electrophoresis was conducted at 4° C. on 10% polyacrylamide gel. The peptide was visualized with FLA-7000 Phosphor Imager (GE Healthcare Life Sciences, USA) and analyzed using the Multi Gauge software.

(2) EMSA of Yeast tRNA or IIB RRE

A peptide (sample), yeast tRNA and IIB RNA were mixed with 10 mM potassium phosphate, pH 7.5, 100 mM NaCl and 10% glycerol. After serially diluting a cell-penetrating peptide prepared in Example 11 (CPP11) or the Tat peptide, RNA solution of the same volume was added. The mixture was incubated at room temperature for 30 minutes and electrophoresis was conducted for 120 minutes on 1% agarose gel under the condition of 4° C. and 100 V. The RNA was visualized with a SYBR Gold nucleic acid gel stain (Invitrogen, USA).

### NMR Analysis

### 1) Sample Preparation and Data Analysis

/Rev NTD (Rev₉₋₆₅) (/Rev) and cRev NTD (Rev₉₋₆₅) (cRev) were prepared in the same manner as unlabeled samples except that expression was performed in ¹⁵N- and ¹³C-concentrated M9 minimal medium for isotopic labeling. All NMR data were measured using Bruker 700, 800 and 900 MHZ NMR spectrometers equipped with cryogenic probes. Backbone resonance assignments of the two proteins were carried out using 2D-[¹⁵N, ¹H] sensitivity-enhanced HSQC (heteronuclear single-quantum coherence) and a suite of 3D triple resonance experiments, HNCA, HNCACB, CBCA (CO) NH and HNCO. The protein samples were dissolved in an NMR buffer at various concentrations (0.15-0.3 mM) and measurement was made at 298 K. The NMR buffer was prepared by mixing 10 mM potassium phosphate, pH 6.0, 150 mM NaCl, 0.1 M EDTA and 10% D20. For side-chain resonance assignments, HNHA, HNHB, HCCH-TOCSY, 2D [¹H, ¹H] NOESY, [¹⁵N, ¹H] NOESY-HSQC and [¹³C, ¹H] NOESY-HSQC were recorded. All ¹H chemical shifts were referenced with respect to the external standard DSS (4,4-dimethyl-4-silapentane-1-sulfonic acid) and ¹³C/¹⁵N chemical shifts were referenced indirectly. Topspin 4.0.3 (Bruker, Germany) and NMRPipe were used for data processing and the spectra were analyzed using CCPN.

### 2) NMR Assignments

Sequence-specific resonance assignments for almost all ¹H, ¹³C and ¹⁵N spins of /Rev and cRev were carried out in the step 1). The spectrum characteristics of Ala, Gly, Ser and Thr were selected using the characteristic ¹³C chemical shift as a starting point for specifying the spectra. In the /Rev protein, most of amide ¹⁵N/¹H (85%) can be specified clearly in addition to the side chains (¹H, ¹³C and ¹⁵N) of amino acid residues except aromatic carbon/proton and proline. In the cRev protein, 97% of amide ¹⁵N/¹H can be specified along with side chains (¹H, ¹³C and ¹⁵N) except aromatic carbon/proton and proline. Resonance frequency could not be assigned to the first residue Gly1 from the N-terminal of /Rev due to the absence of peaks owing to exchange or overlap and rapid exchange of water with Arg38, Arg39, Trp40, Arg41, Glu42 and Arg43. The ¹H, ¹³C and ¹⁵N resonance of /Rev was assigned using HNCO, HNCA, HNCACB, CBCA (CO) NH and 2D [¹H-¹H] NOESY. The two proteins contained three Asn residues for assignment of side-chain NH₂. While the two proteins contained four Gln residues, the side-chain NH₂ of three Gln residues was assigned for /Rev and the side chains of all Gln residues were assigned for cRev. In total, 86.69% of all backbones (254 out of 293), 65.23% of side-chain protons (167 out of 256) and 50.92% of side-chain carbons and nitrogens (111 out of 218) were assigned for/Rev. Similarly, 96.93% of all backbones (316 out of 326), 78.30% of side-chain protons (220 out of 281) and 59.24% of side-chain carbons and nitrogens (141 out of 238) were assigned for cRev. Since only cRev contains one Cys residue with a ¹³C^{β} chemical shift occurring at 28.08 ppm, it can be predicted that it is in reduced form. The complete backbone assignments for the two proteins are shown in [¹⁵N, ¹H]-HSQC. The chemical shifts assigned to the two proteins were registered in BioMagResBank (http://www.bmrb.wisc.edu) with accession codes 36488 and 36486, respectively.

### Structure Calculation

The solution structure of the proteins was calculated using CYANA3.97 from NMR-derived distance and torsion angle restraints. The minimum and maximum distances were set to 2.4 Å and 6.4 Å, respectively, in automatic calculation. For the peaks that could not be integrated accurately, distance was classified as strong (1.8-2.5 Å), intermediate (1.8-3.5 Å), weak (1.8-5.0 Å) or very weak (1.8-6.4 Å). The peaks of 3D ¹⁵N- and ¹³C-edited NOESY spectra (mixing time (τₘ)=120 ms) were selected using CCPN and the information derived therefrom was used as an input to CYANA3.97. The HNHA spectrum was used to measure ³J (H^{N}-HA) coupling to estimate the backbone ϕ torsion angles for the peptides. Other dihedral angles were obtained from TALOS chemical shift analysis. The restraint ranges for the dihedral angles derived from TALOS were used to determine the estimate deviation. The distance and dihedral angle constraints used for the structure calculation of the two proteins are summarized in Table 2. The structure calculation was performed using a standard simulated annealing protocol with 20,000 torsion angle dynamics steps from 200 random conformers. In the final execution step, 20 structures with the lowest residual target function values were selected for further analysis. The RMSD (root-mean-square deviation) values of the backbone and all atoms were calculated using CYANA3.97 and PyMOL (www.pymol.org). The quality of the final structures was confirmed using PROCHECK and protein structure verification softwares. The 20 linear and cyclic structures were submitted to the Protein Data Bank (PDB) with submission numbers (PDB IDs) 8X3P (/Rev) and 8X30 (cRev), respectively.

TABLE 2
Conformationally restricting restraints
Distance Restraint List

| | Linear¹ | Cyclic² |
|---|---|---|
| Total | 251 | 196 |
| Intraresidue ((\|i − j] = 0) | 111 | 96 |
| Sequential (\|i − j\| = 1) | 139 | 84 |
| Medium-range (1 < \|i − j\| < 5) | 1 | 8 |
| Long-range (\|i − j \| ≥ 5) | 0 | 8 |
| Hydrogen bonds | 0 | 0 |
| Dihedral angle restrains (f and j) | 14 | 44 |
| Disulfide restraints | 0 | 0 |
| No. of restraints per residue | 4.5 | 3.7 |

Residual restraint violations

| | | |
|---|---|---|
| Average no. of distance violations | 0 | 0 |
| per structure > 0.2 Å | | |
| Average no. of dihedral angle violations | 0 | 0 |
| per structure >3° | | |
| Average no. of van der Waals violations | 0 | 0 |
| per structure >0.2 Å | | |

Model quality

| | | |
|---|---|---|
| Rmsd backbone atoms (Å) | 0.303 | 0.704/0.375 |
| Rmsd heavy atoms (Å) | 1.033 | 1.454/1.495 |
| Rmsd bond lengths (Å) | 0.001 | 0.001 |
| Rmsd bond angles (0) | 0.2 | 0.2 |

MolProbity Ramachandran statistics

| | | |
|---|---|---|
| Most favored region (%) | 65.7 | 72.5 |
| Allowed region (%) | 34.0 | 27.3 |
| Additionally allowed region (%) | 0.1 | 0.0 |
| Disallowed region (%) | 0.2 | 0.2 |

Global quality scores (raw/Z score)

| | | |
|---|---|---|
| Verify3D | −0.08/−8.67 | 0.01/−7.22 |
| PROCHECK ( [symbol] - y) | 0.02/0.39 | 0.43/2.01 |
| PROCHECK (all) | −0.23/−1.36 | 0.01/0.06 |
| MolProbity clash score | 0.93/1.37 | 1.55/1.26 |
| Model contents | | |
| Total no. of residues | 59 | 65 |
| BMRB accession number | 36488 | 36486 |
| PDB ID code | 8X3P | 8X3O |

### Fluorescence Anisotropy Analysis

IIB RRE labeled with cRev and Cy3 was dissolved in a binding buffer (25 mM sodium phosphate, 50 mM NaCl, pH 6.0). The peptide (sample) of various concentrations was sequentially added to 250 nM RNA. The final concentration of the peptide was 600 nM. After the mixing, the sample was incubated for 1 minute. There was 1-minute incubation time between all the steps. All the experiments were performed at room temperature. The dissociation constant (K_{d}) was calculated using the Origin software.

### Cytotoxicity Analysis

Cytotoxicity was analyzed using the WST-8 (5-(2,4-disulfophenyl)-3-(2-methoxy-4-nitrophenyl)-2-(4-nitrophenyl)-2H-tetrazolium, water-soluble tetrazolium). First, HeLa cells were inoculated onto a 96-well plate (5,000 cells/well) and cultured for 24 hours in a DMEM medium containing 10% FBS. After washing with PBS, the peptide (sample) of various concentrations was added to Opti-MEM. 24 hours later, the WST-8 reagent was added and incubation was conducted for 1 hour. Then, WST-8 formazan was quantified using a Victor X5 multilabel plate reader (Perkin Elmer, USA).

### Flow Cytometry

Cells (Hela, HCT116 and 293T cells) were inoculated onto a 24-well plate (10⁵ cells/well) and cultured for 24 hours in a DMEM medium containing 10% FBS. After washing with PBS, the cells were treated with the fluorescence-labeled peptide (sample) for 4 hours. Then, after washing twice with PBS, the cells were incubated with trypsin (1 mg/mL) for 15 minutes and the peptide bound to the cell surface was removed. The cells were centrifuged at 1,200 rpm for 5 minutes, washed twice with PBS and then suspended in a PBS buffer containing 10% non-enzymatic cell dissociation solution. The fluorescence intensity of the internalized peptide was measured using a BD LSR II flow cytometer (BD Biosciences, USA).

Treatment with Endocytosis Inhibitor

Hela cells were inoculated onto a 24-well plate (2×10⁵ cells/well) and cultured for 24 hours in a DMEM medium containing 10% FBS. Half of the cultured cells were used in experiment wherein endocytosis was inhibited by treating with chemical inhibitors and the remaining half were used in experiment wherein endocytosis was inhibited by lowering temperature. After washing with PBS, the cells were treated with the endocytosis inhibitors amiloride (20 μM) or dynasore (50 M) for 1 hour or incubated at 4° C. for 1 hour. The cells were treated with the peptide (sample) for 30 minutes in an Opti-MEM (30 μM) medium and then analyzed by flow cytometry.

### Confocal Microscopy

A DMEM medium containing 10% FBS and an 8-well Lab-Tek II chamber slide (Thermo Fisher Scientific, USA) were prepared. Hela cells were dispensed into each well at a density of 10⁴ cells/well and cultured for 24 hours. After washing the cells twice with PBS, the cells were cultured for 30 minutes after replacing the medium with a fresh one containing FAM-labeled peptide (sample) (10 μM). The cultured cells were washed 5 times with PBS. The living cells were visualized using an LSM 880 confocal microscope (Carl Zeiss, Germany).

### In-Vivo Fluorescence Imaging

All animal experimental procedures including sample administration and euthanasia were performed in accordance with the strict guidelines of the Yonsei University Health System Institutional Animal Care and Use Committee. Female 7-week BALB/c nude mice were purchased from Orient Bio Company (Korea). Fluorescence-labeled peptide (sample) (10 mg/kg) was injected via the lateral tail vein of the mice. Fluorescence images were acquired at different times (0, 4, 8 and 12 hours) using an in-vivo imaging system (IVIS; PerkinElmer, USA) and the images were analyzed with the Living Image software (PerkinElmer).

### Examples and Test Examples

### Preparation Examples 1 and 2. Preparation of /Rev NTD (Rev₉₋₆₅) (/Rev) and cRev NTD (Rev₉₋₆₅) (cRev)

In order to analyze the mechanism of Rev ARM-IIB binding and the possibility of conformational selection by cyclization of the Rev protein, the structural difference in the linear Rev protein (hereinafter, referred to as ‘/Rev’) and the cyclic Rev protein (hereinafter, referred to as ‘cRev’) was investigated.

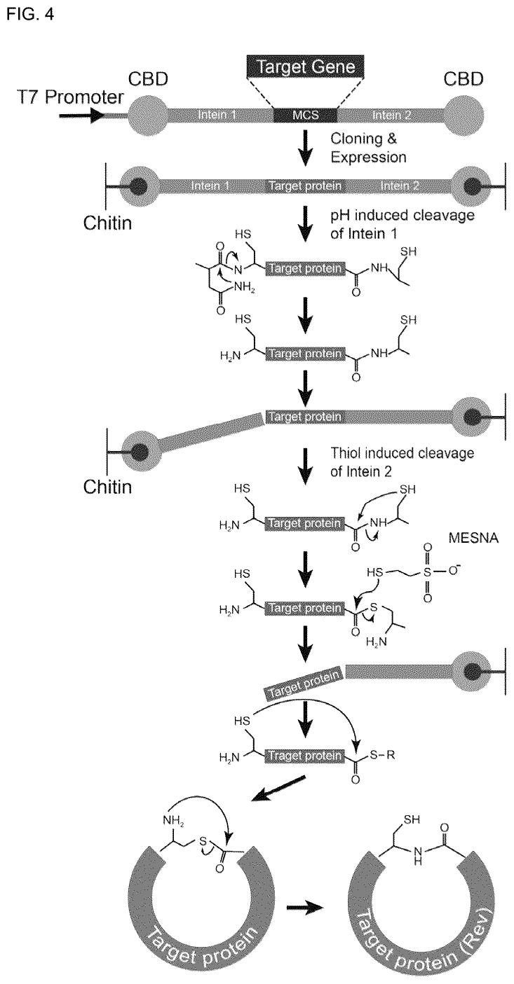

For this, /Rev NTD (Rev₉₋₆₅) and cRev NTD (Rev₉₋₆₅) were prepared. cRev NTD (Rev₉₋₆₅) was designed by replacing the important hydrophobic residues involved in protein oligomerization (Leu-12, Val-16, Leu-18, Ile-55 and Leu-60) with Ala in order to eliminate the oligomerization which makes the analysis of the structural state of the Rev protein difficult. The result is shown in FIG. 3 and FIG. 4 and sequences are given in Table 3.

cRev was prepared by cyclization after expressing with bacteria according to the intein-mediated protein ligation method (Xu, M. Q. & Evans, T. C., Jr. Intein-mediated ligation and cyclization of expressed proteins. Methods 24, 257-77 (2001)).

The protein expression processes for /Rev NTD (Rev₉₋₆₅) and cRev NTD (Rev₉₋₆₅) are as follows. First, /Rev was expressed in the E. coli strain BLR (DE3) using a pET-28a vector containing a cleavable His-tag, a GB1 domain and a TEV protease cleavage site at the N-terminal. The cells were cultured at 37° C. in an LB medium containing 30 μg/mL kanamycin. The cells were cultured until OD₆₀₀ reached 0.6. Then, after adding 0.5 mM IPTG (isopropyl-β-D-thiogalactopyranoside) to the medium, expression was induced at 25° C. for 12 hours. After centrifuging the culture at 4000×g for 10 minutes, the harvested cells were treated with a lysis buffer (20 mM Tris-CI pH 7.4, 500 mM NaCl, 10 mM imidazole). His-tag-GB1-/Rev was purified from the lysate by Ni-NTA affinity chromatography and the GB1 domain and the His-tag were cleaved with TEV protease. The cleaved /Rev was purified by RP-HPLC.

cRev NTD (Rev₉₋₆₅) (cRev) was expressed in the E. coli strain Rosetta (DE3) pLysS using a pTWIN2 vector (NEB, USA). The cRev gene designed as described above was cloned between the intein gene using the Sap I restriction site. After the transformation, the cells were cultured at 37° C. in an LB medium containing 100 μg/mL ampicillin. The cells were cultured until OD₆₀₀ reached 0.5. After adding 1 mM IPTG to the cell culture, expression was induced at 16° C. for 16 hours. The cells were harvested and resuspended in a lysis buffer (20 mM Na-HEPES pH 8.5, 500 mM NaCl, 1 mM EDTA). The cell lysate was centrifuged at 10,000×g for 1 hour and loaded into a chitin column at a flow rate of 1 mL/min. Then, unreacted impurities were removed with 100 mL of a lysis buffer.

On-column cleavage of intein 1 was induced by equilibrating beads with a buffer containing 20 mM Na-HEPES, pH 6.5, 500 mM NaCl and 1 mM EDTA. After incubation at room temperature overnight, impurities were removed by washing the column with a lysis buffer. After inducing second on-column cleavage of intein 2 by equilibrating the beads with a buffer containing 20 mM HEPES, pH 8.5, 500 mM NaCl, 50 mM 2-mercaptoethanesulfonic acid and 1 mM EDTA, cyclization was performed. The column was incubated overnight at 4° C. and then cRev was eluted from the column. The fractions containing cRev were further purified by RP-HPLC.

TABLE 3

| | | |
|---|---|---|
| /Rev | GGSDEDALKAARAIKFLYQSNPPPNPEGTR | SEQ ID NO 36 |
| | QARRNRRRRWRERQRQIHSISERILSTYL | |
| Rev | CRGGDEDALKAARAIKFLYQSNPPPNPEGT | SEQ ID NO 37 |
| wild | RQARRNRRRRWRERQRQIHSISERILSTLG | |
| type | GMRM | |
| cRev | CRGGDEDALKAARAIKFLYQSNPPPNPEGT | SEQ ID NO 38 |
| | RQARRNRRRRWRERQRQIHSASERIASTLG | |
| | GMRM | |

In order to confirm whether the cRev and /Rev proteins were synthesized properly through the processes described above, SDS-PAGE and MALDI-TOF MS analyses were conducted after the HPLC purification.

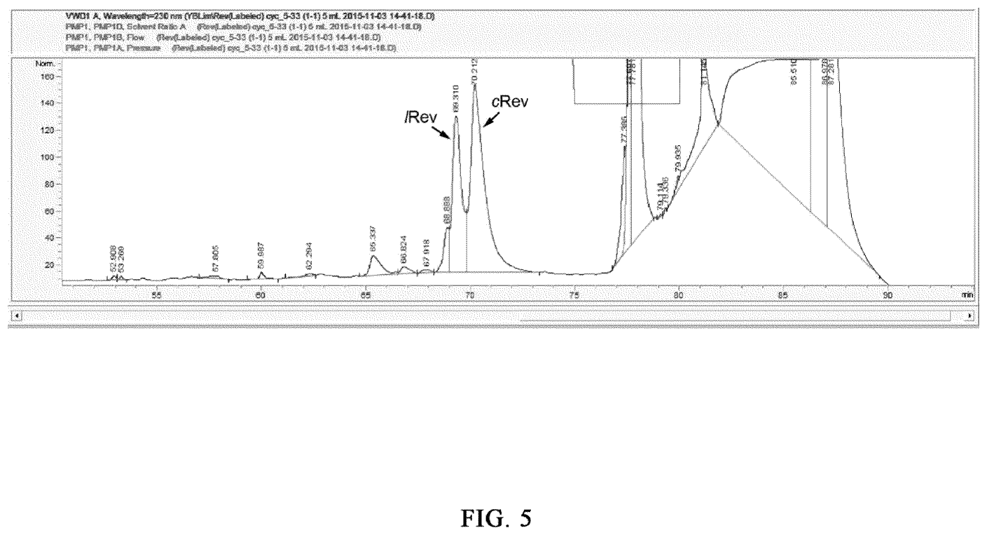

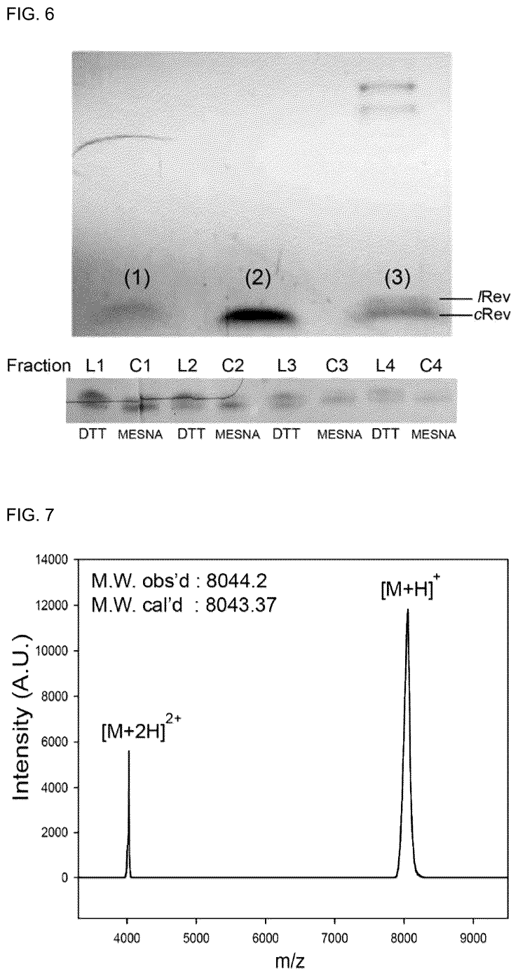

FIGS. 5-7 show the analysis results for the cRev and /Rev proteins. FIG. 5 shows the analysis result for the proteins purified by HPLC, FIG. 6 shows the SDS-PAGE analysis result, and FIG. 7 shows the MALDI-TOF MS analysis result of cRev. From FIGS. 5-7, it can be seen that the two proteins (/Rev and cRev) were synthesized successfully.

### Examples 1-23. Synthesis of Helical Cell-Penetrating Peptides (CPPα)

23 peptides described in Table 4 were synthesized by Fmoc-based solid-phase peptide synthesis using Rink Amide resin. Specifically, Fmoc-protected amino acids (5 equiv), HOBt (4.5 equiv) and DIPEA (10 equiv) were added to the resin suspended in a DMF solvent. Then, protein synthesis was performed according to the standard Fmoc protocol using a Tribute peptide synthesizer (Protein Technologies, USA).

Fluorophore-labeled peptides were prepared as needed. FAM (5 (6)-carboxyfluorescein) or TAMRA (5 (6)-carboxytetramethylrhodamine) was used as the fluorophore. The fluorophore was coupled to the terminal of the peptide by treating the peptide-resin complex with the fluorophore (FAM or TAMRA) (5 equiv), HATU (4.5 equiv) and DIPEA (10 equiv).

The synthesized peptide was separated from the resin and the peptide-loaded resin was treated with a cleavage cocktail (TFA:TIS:water=95:2.5:2.5) for 2 hours to remove the standard amino acid-protecting groups, followed by filtration and concentration. The concentrate was treated with TBME (tert-butyl methyl ether) to precipitate the crude peptide. After recovering the precipitate, the peptide was purified finally by RP-HPLC (reversed-phase high-performance liquid chromatography) using a semi-preparative column (I.D.×L; 10×250 mm). Water (0.1% TFA) and acetonitrile (0.1% TFA) was used as eluents and the flow rate of the eluents was set to 2 ml/min.

TABLE 4

| | | | Number of |
|---|---|---|---|
| | Sequence (N → C) | SEQ ID NO | amino acids |
| Example 1 | RQARRNRQARRNRQARRNRQARRN | SEQ ID NO 11 | 24 |
| (CPP1) | | | |
| Example 2 | RQARRNRQARRNRQARRNRQARRNRQARRN | SEQ ID NO 12 | 30 |
| (CPP2) | | | |
| Example 3 | RQARRNRQARRNRQARRNRQARRNRQARRNRQ | SEQ ID NO 13 | 36 |
| (CPP3) | ARRN | | |
| Example 4 | AUAUARQARRNRQARRNRQARRNAUAUA | SEQ ID NO 14 | 28 |
| (CPP4) | | | |
| Example 5 | UAURQARRNUAURQARRNUAURQARRNUAURQ | SEQ ID NO 15 | 39 |
| (CPP5) | ARRNUAU | | |
| Example 6 | QARRNQARRNQARRNQARRN | SEQ ID NO 16 | 20 |
| (CPP6) | | | |
| Example 7 | QARRNQARRNQARRNQARRNQARRN | SEQ ID NO 17 | 25 |
| (CPP7) | | | |
| Example 8 | UQARRNUQARRNUQARRNUQARRNU | SEQ ID NO 18 | 25 |
| (CPP8) | | | |
| Example 9 | UAQARRNUAQARRNUAQARRNUAQARRNUA | SEQ ID NO 19 | 30 |
| (CPP9) | | | |
| Example 10 | UAUQARRNUAUQARRNUAUQARRNUAU | SEQ ID NO 20 | 27 |
| (CPP10) | | | |
| Example 11 | UAUQARRNUAUQARRNUAUQARRNUAUQARRN | SEQ ID NO 21 | 35 |
| (CPP11) | UAU | | |
| Example 12 | AAAQARRNAAAQARRNAAAQARRNAAAQARRN | SEQ ID NO 22 | 35 |
| (CPP12) | AAA | | |
| Example 13 | UAUQARRNUAUQARRNUAUQARRNUAUQARRN | SEQ ID NO 23 | 43 |
| (CPP13) | UAUQARRNUAU | | |
| Example 14 | UaUgarrnUaUqarrnUaUqarrnUaUqarrn | SEQ ID NO 24 | 35 |
| (CPP11d) | UaU | | |
| Example 15 | UAUQRARNUAUQRARNUAUQRARNUAUQRARN | SEQ ID NO 25 | 35 |
| (CPP14) | UAU | | |
| Example 16 | UAUQRRANUAUQRRANUAUQRRANUAUQRRAN | SEQ ID NO 26 | 35 |
| (CPP15) | UAU | | |
| Example 17 | UAUNRRAQUAUNRRAQUAUNRRAQUAUNRRAQ | SEQ ID NO 27 | 35 |
| (CPP16) | UAU | | |
| Example 18 | UAUNRARQUAUNRARQUAUNRARQUAUNRARQ | SEQ ID NO 28 | 35 |
| (CPP17) | UAU | | |
| Example 19 | UAUNARRQUAUNARRQUAUNARRQUAUNARRQ | SEQ ID NO 29 | 35 |
| (CPP18) | UAU | | |
| Example 20 | UAUQARRNUAUQARRNUAUQARRNUGUQARRN | SEQ ID NO 30 | 35 |
| (CPP19) | UAU | | |
| Example 21 | UAUQARRNUAUQARRNUGUQARRNUGUQARR | SEQ ID NO 31 | 35 |
| (CPP20) | NUAU | | |
| Example 22 | UAUQARRNUGUQARRNUGUQARRNUGUQARR | SEQ ID NO 32 | 35 |
| (CPP21) | NUAU | | |
| Example 23 | AAAQARRNAAAQARRNAAAQARRNAAAQARRN | SEQ ID NO 33 | 43 |
| (CPP22) | AAAQARRNAAA | | |

In Table 4, the repeating sequences were underlined and D-amino acids were written in lowercase letters. Aib (2-aminoisobutyric acid) was denoted as ‘U’.

### Test Example 1-1. Designing of Cell-Penetrating Peptide from Rev Protein

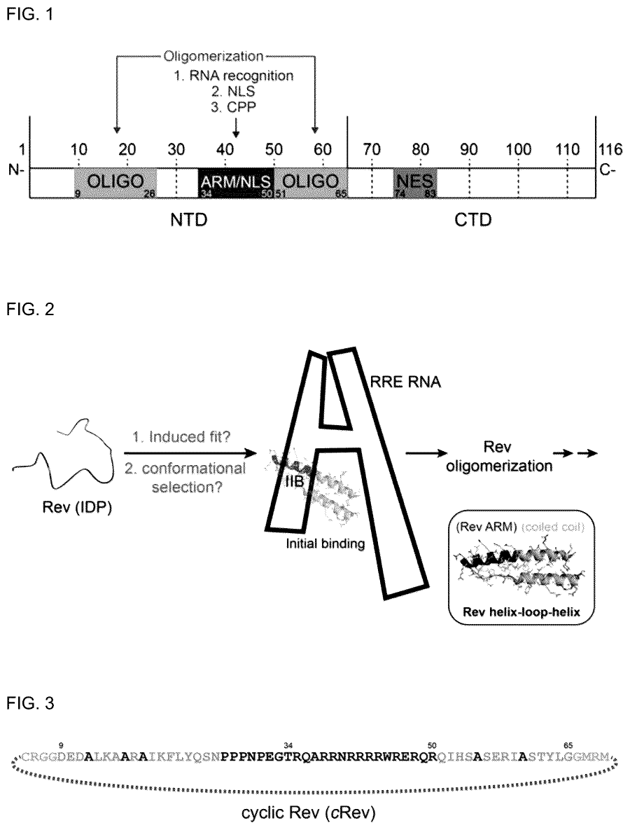

FIG. 1 schematically shows the domain structure of Rev, wherein OLIGO represents the oligomerization domain, NES represents the nuclear export signal, NTD represents the N-terminal domain and CTD represents the C-terminal domain. FIG. 2 schematically shows the binding mechanism of the Rev protein and RRE RNA. The insert in FIG. 2 shows the structure of Rev NTD (PDB 2X7L) after binding of the Rev protein to the IIB region of RRE RNA.

Referring to FIG. 1 and FIG. 2, the binding mechanism of the HIV-1 Rev protein to RRE RNA was analyzed to design a new cell-penetrating peptide.

First, the HIV-1 Rev protein plays a key role in viral replication because this protein is responsible for the nucleocytoplasmic export of viral mRNAs. Intrinsically disordered Rev folds upon complex formation with RRE RNA, in which only residues 9 to 65 in the N-terminal domain (NTD) become ordered upon RNA binding. The initial high-affinity binding of Rev occurs at the stem-loop IIB region of RRE. The ordered Rev₉₋₆₅ adopts an asymmetric helix-loop-helix (HLH) motif.

After the initial Rev binding, additional protein-protein interactions between the ordered oligomerization domains (OLIGOs) and protein-RNA interactions at low-affinity sites cooperatively oligomerize an additional 7-9 Rev molecules on an overall A-shaped RRE (˜350 nt). The oligomerized ribonucleoprotein (RNP) is then competent for nucleocytoplasmic export. When the Rev and viral mRNA are released from the RNP in the cytosol, Rev is imported back into the nucleus for recycling.

Interestingly, an arginine-rich motif (ARM) of Rev (residues 34-50) performs at least three functions, namely, RNA recognition, nuclear localization signal (NLS) function, and cell-penetrating peptide (CPP) activity.

In summary, as the disordered Rev ARM (Rev34-50) motif binds to IIB (e.g., coil-to-helix transition) to form a complex, conformational change occurs to form a nearly perfect α-helix structure. However, it has not yet been reveled clearly whether the binding of the disordered Rev and RRE RNA occurs via the conformational selection model or the induced fit model.

But, based on the fact that the Rev ARM motif is partially observed as a helix from the CD (circular dichroism) analysis result, it is predicted that the binding of the Rev protein and RRE RNA will be governed by the conformational selection model.

According to the recent NMR structural research on the Rev mutant having defects in the Rev ARM motif and the oligomerization site (oligomerization-defective full-length Rev mutant), the Rev ARM motif of the Rev mutant has a random coil structure in solution and forms binding according to the induced fit mechanism (Casu, F., Duggan, B. M. & Hennig, M. The arginine-rich RNA-binding motif of HIV-1 Rev is intrinsically disordered and folds upon RRE binding. Biophys J 105, 1004-17 (2013)). The ARMs of the conventional RNA-binding proteins such as HIV-1 Tat and BIV (bovine immunodeficiency virus) Tat are also known to exist in disordered structures with no secondary structures in free state and bind to the same type of RNAs via the induced fit model.

It is known that the cyclization of peptides provides molecular constraint, which increases the structural stability of the peptides and proteins by reducing the structural entropy of the unfolded state.

Taking the above information into account, /Rev NTD (Rev₉₋₆₅) (hereinafter, referred to as ‘/Rev’) and cRev NTD (Rev₉₋₆₅) (hereinafter, referred to as ‘cRev’) were designed from the Rev protein and their structures were analyzed.

### Test Examples 1-2. Structural Analysis of /Rev and cRev

The structural characteristics of the /Rev and cRev prepared in Preparation Examples 1 and 2 were compared by SEC (size exclusion chromatography) analysis. The SEC analysis was performed using a Superdex peptide column with 300 mM NaCl as a mobile phase. For comparison, carbonic anhydrase, cytochrome C and aprotinin were also analyzed.

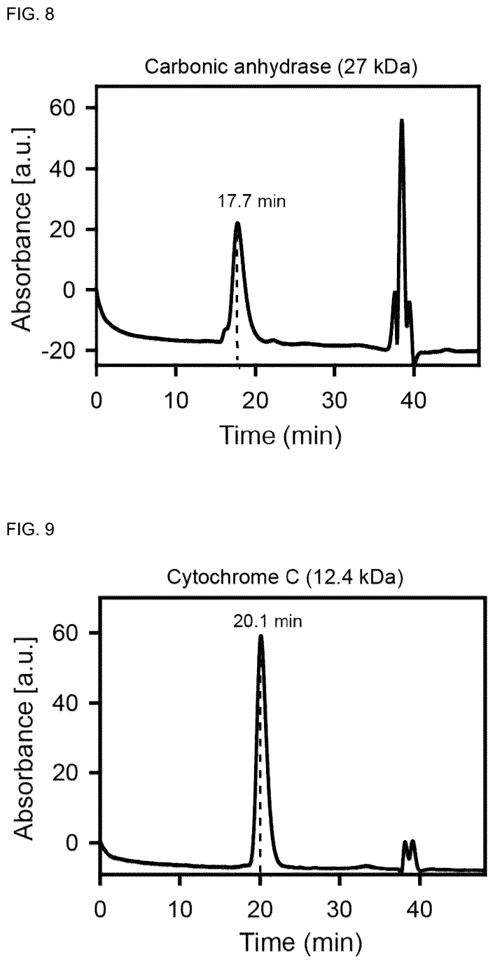

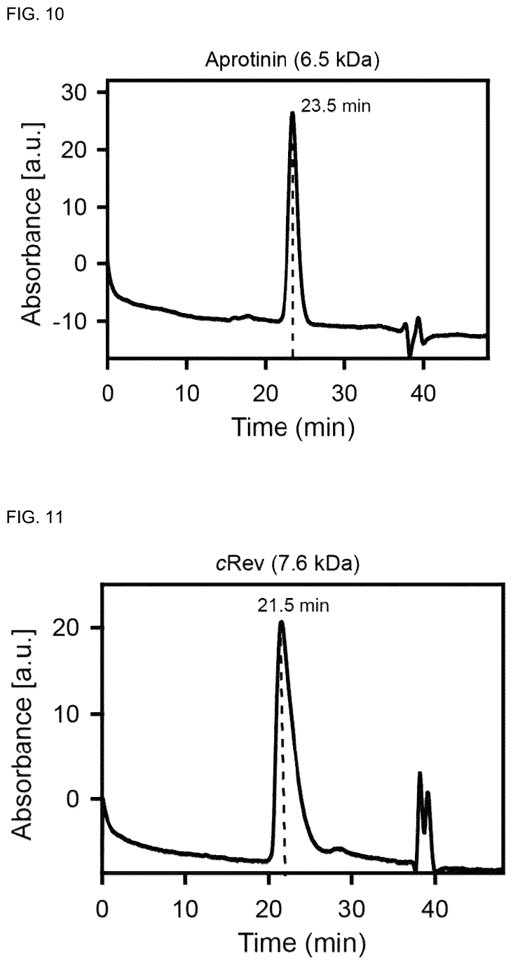

FIG. 8 shows the SEC (size-exclusion chromatography) analysis result for carbonic anhydrase, FIG. 9 shows the SEC (size-exclusion chromatography) analysis result for cytochrome C, FIG. 10 shows the SEC (size-exclusion chromatography) analysis result for aprotinin, FIG. 11 shows the SEC (size-exclusion chromatography) analysis result for cRev NTD (Rev₉₋₆₅), and FIG. 12 shows the SEC (size-exclusion chromatography) analysis result for /Rev NTD (Rev₉₋₆₅).

As shown in FIGS. 8-12, it was confirmed that /Rev and cRev were prepared successfully as monomers without oligomerization or aggregation.

### Test Example 2. Comparative Analysis of Structural Characteristics of /Rev and cRev

The /Rev and cRev prepared in Preparation Examples 1 and 2 were analyzed by CD spectroscopy.

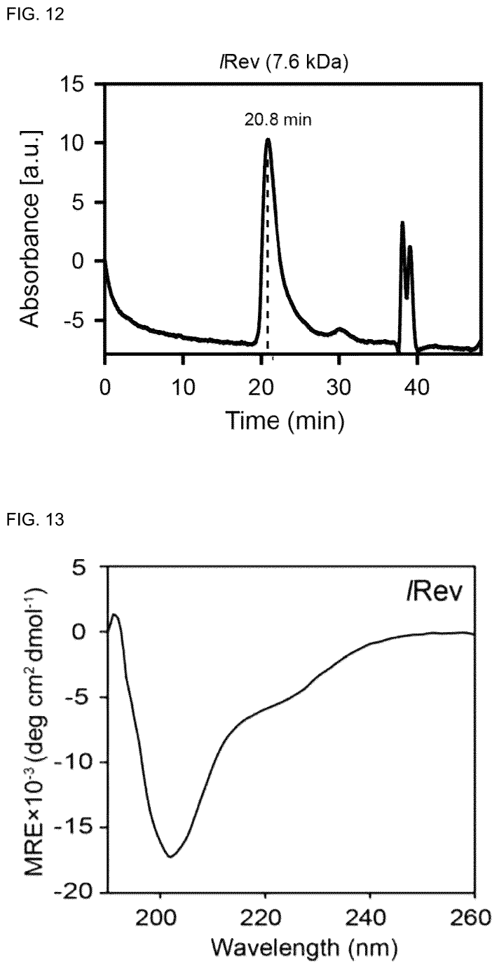

FIG. 13 shows the CD spectrum of /Rev and FIG. 14 shows the CD spectrum of cRev. It was confirmed that /Rev and cRev have random coil structures. However, a weak band indicating the α-helix structure was identified at about 222 nm fin both spectra.

A complex was prepared by mixing the cRev prepared in Preparation Example 2 with IIB RNA and it was analyzed by CD spectroscopy. The mixing ratio of cRev/IIB was 1. The complex sample was dissolved in 10 mM potassium phosphate, 10 mM NaCl and 0.1 μM EDTA and then analyzed under the condition of pH 6.0 and 25° C.

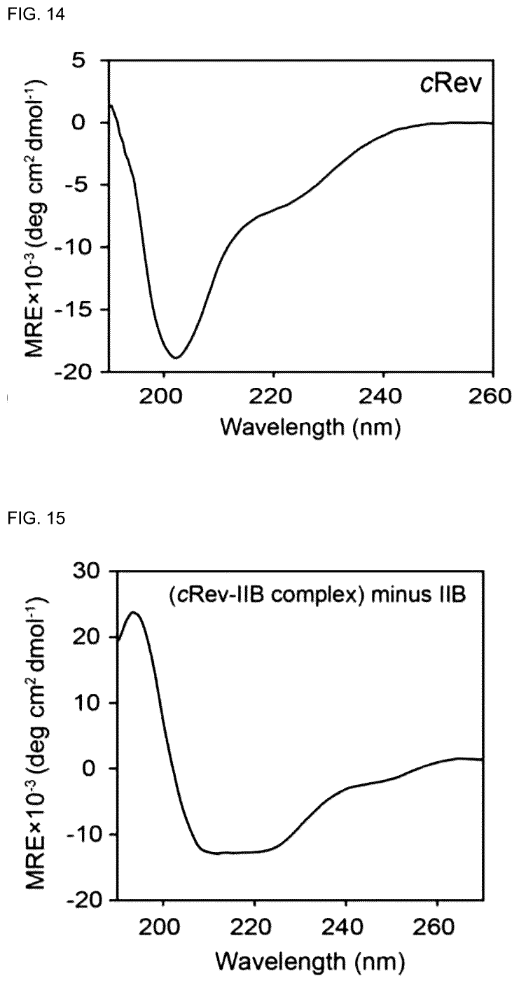

FIG. 15 shows the CD spectrum of the cRev-IIB RNA complex. Since the Rev NTD partially contains Rev ARM and OLIGOs, it was expected that coil-to-helix transition would occur in the Rev ARM fragment during or after the completion of the IIB binding process and, as a result, a coiled coil structure would be formed in the OLIGO fragment through helix nucleation and propagation. Indeed, it was confirmed that cRev forms an almost perfect α-helix through structural changes upon binding to IIB RNA.

A complex (cRev-IIB complex) was prepared by mixing the cRev prepared in Preparation Example 2 with IIB RNA and UV melting experiment was performed. The cRev/IIB ratio was 1. For the control group (IIB RNA only), only IIB RNA was used without cRev.

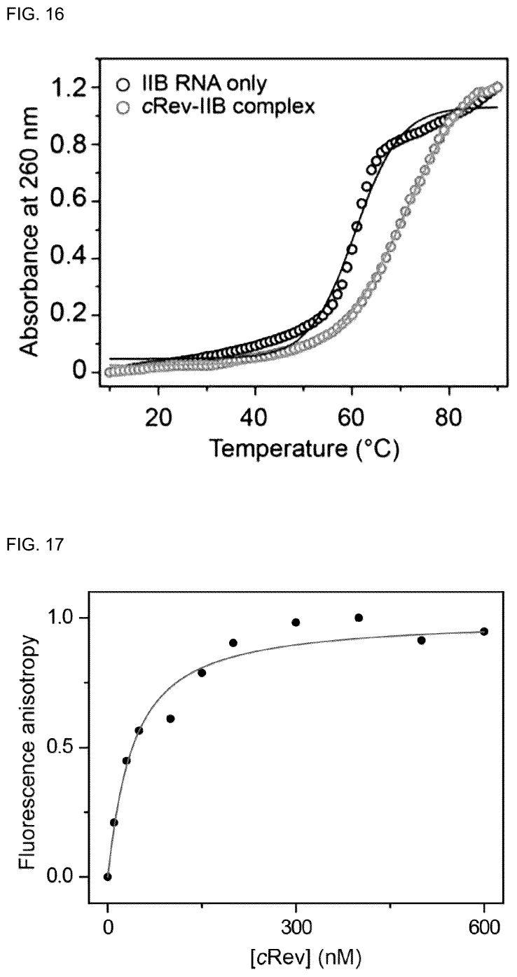

FIG. 16 shows the UV melting curves of IIB RNA and the cRev-IIB complex. The Tₘ of the IIB RNA only sample was 60.6° C. and the Tₘ of the cRev-IIB complex was 70.8° C. It was confirmed that cRev forms strong protein-RNA interaction upon binding to IIB RNA and thermal stability is increased significantly as compared to IIB RNA only.

### Test Example 3. Functional Analysis of cRev Protein

In order to analyze the function of cRev, the cRev of Preparation Example 2 was mixed with Cy3-labeled IIB RNA and then fluorescence anisotropy analysis was performed.

FIG. 17 shows the fluorescence anisotropy analysis result for the mixture of cRev and Cy3-IIB RNA.

As shown in FIG. 17, cRev was confirmed to have K_{d} (dissociation constant) of 34 nM for the interaction with IIB RNA.

### Test Example 4. Binding Characteristics of cRev Protein for IIB RNA

In order to analyze the binding affinity of cRev for IIB RNA, the /Rev of Preparation Example 1 or the cRev of Preparation Example 2 was mixed with ³²P-labeled wild-type IIB and ³²P-labeled mutant IIB, respectively, and analyzed by EMSA (electrophoretic mobility shift assay). The mutation of IIB is the mutation in the G46-C74 base pairs of wild-type IIB.

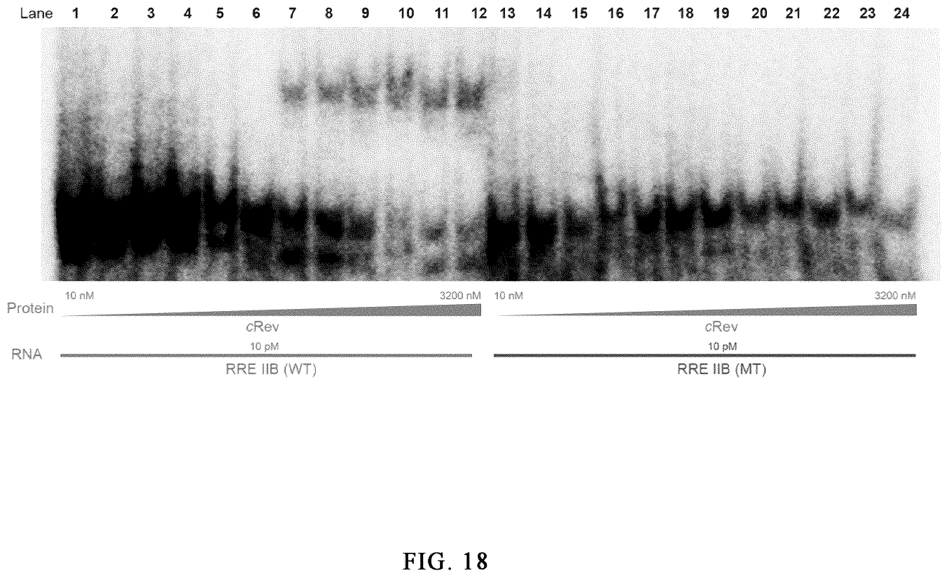

FIG. 18 shows the EMSA (electrophoretic mobility shift assay) result for a mixture of the cRev of Preparation Example 2 and wild-type IIB (RRE IIB WT) or mutant IIB (RRE IIB MT).

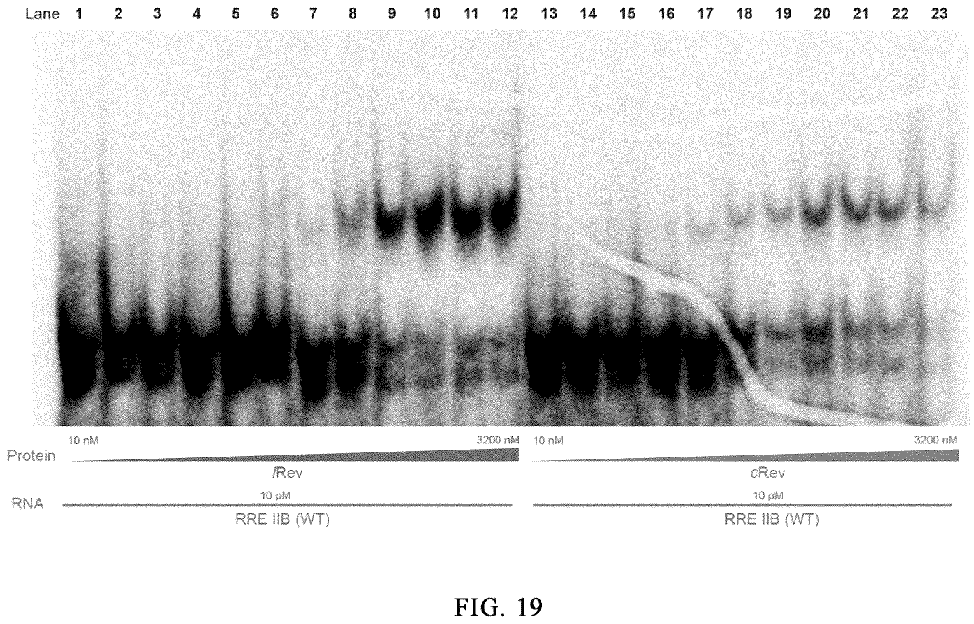

FIG. 19 shows the EMSA (electrophoretic mobility shift assay) result for a mixture of the cRev of Preparation Example 2 or the /Rev of Preparation Example 1 and wild-type IIB (RRE IIB WT).

As shown in FIG. 18 and FIG. 19, the cRev of Preparation Example 2 specifically recognizes IIB RNA. The cRev had significantly higher binding affinity than the /Rev of Preparation Example 1, indicating that the structure of the Rev protein can be changed into a more favorable form for the recognition of IIB RNA through cyclization.

### Test Example 5. Analysis of Solution Structure of cRev Protein or /Rev Protein

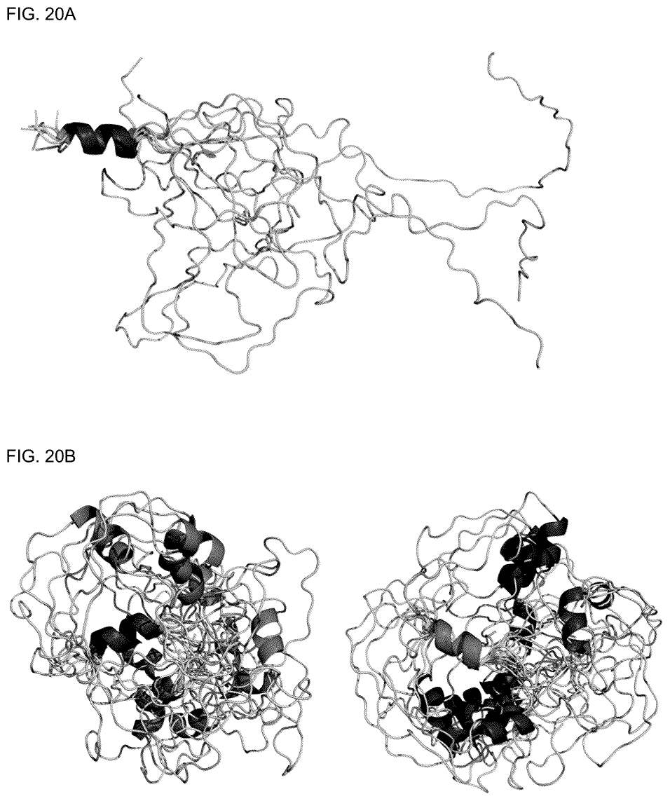

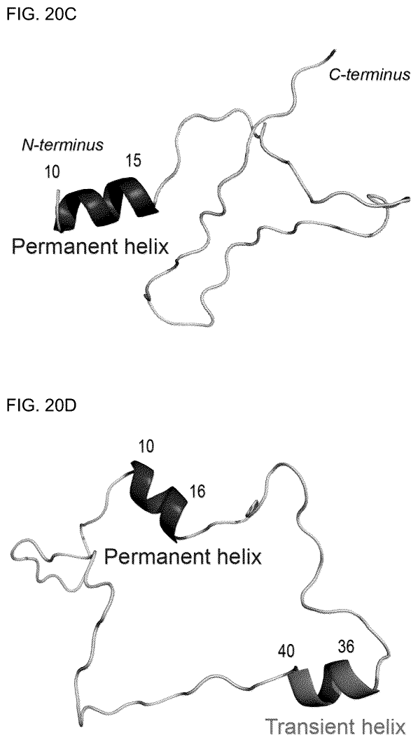

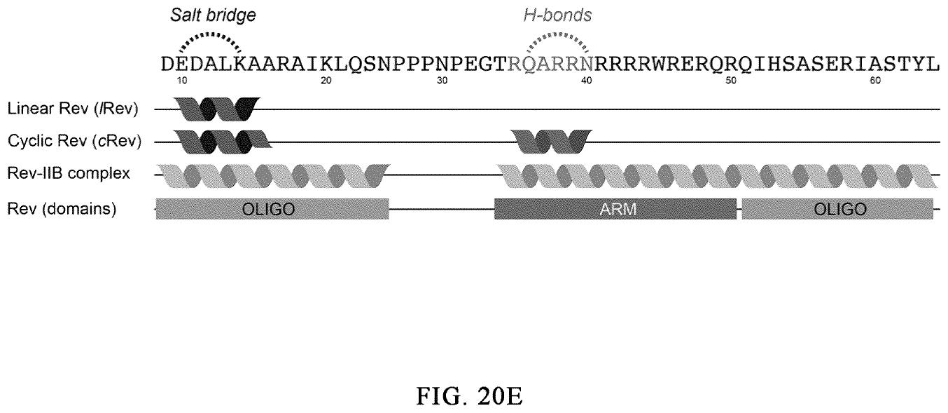

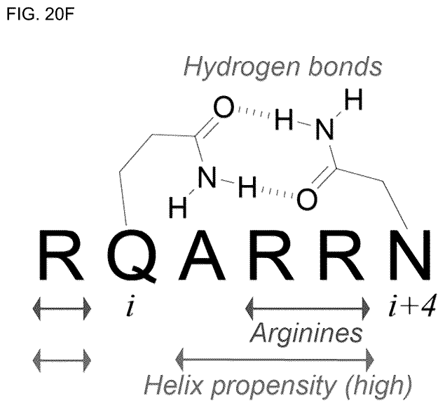

In order to understand the structural difference of the cRev of Preparation Example 2 or the /Rev of Preparation Example 1, NMR solution structure was analyzed using ¹³C- and ¹⁵N-labeled proteins. The result is shown in FIG. 20.

FIGS. 20A to 20F show the NMR solution structure of the cRev of Preparation Example 2 and the /Rev of Preparation Example 1 at 298 K. FIG. 20A shows a set of 20 conformers exhibiting the solution structure of /Rev NTD (Rev₉₋₆₅) (/Rev) (PDB 8X3P) and FIG. 20B shows a set of 20 conformers exhibiting the solution structure of cRev NTD (Rev₉₋₆₅) (cRev) (PDB 8X30). FIG. 20C shows the monomer structure of /Rev NTD (Rev₉₋₆₅) (/Rev) and FIG. 20D shows the monomer structure of cRev NTD (Rev₉₋₆₅) (cRev). FIG. 20E schematically compares the secondary structure elements of /Rev NTD (Rev₉₋₆₅) (/Rev), cRev NTD (Rev₉₋₆₅) (cRev) and the Rev protein in the Rev-IIB complex. FIG. 20F shows the sequence analysis result of an alpha-helix fragment which is critical in the Rev protein.

As shown in FIG. 20A, it was confirmed that the conformers of the /Rev protein overlap based on the N-terminal helix (blue).

As shown in FIG. 20B, it was confirmed that the conformers of the cRev protein overlap based on the N-terminal helix (blue) and the C-terminal helix (red).

As shown in FIG. 20A and FIG. 20C, it can be seen that the structure of the/Rev protein of Preparation Example 1 is disordered except for the presence of the short α-helix at the N-terminal. In contrast, as shown in FIG. 20B and FIG. 20D, it can be seen that the cRev protein of Preparation Example 2 has two helical domains at both the N- and C-terminals. The helix structure can be confirmed through a weak but identifiable band for the α-helix in the CD spectrum. As shown in FIG. 20E, it can be seen that helix at the N-terminal is a part of a coiled coil in OLIGO.

The increased ability to form a helix at the N-terminal in the cRev of the present disclosure may be considered to be due to alanine substitution (L12A and L16A). Considering the similar helix propensity of leucine and alanine, the possibility that a helix exists in wild-type Rev should be very high. But, the helix near the C-terminal was confirmed to be stabilized only in cRev. That is to say, it can be seen that the molecular constraint of cyclization increases the stability for the C-terminal helix structure as the unfolding rate of cRev decreases and that alanine substitution (L12A and L16A) has no effect. In the non-cyclized, linear /Rev of Preparation Example 1, no helix structure was observed near the C-terminal.

The sequences of the N-terminal helix and the C-terminal helix were analyzed as shown in FIG. 20E. As a result, it was confirmed that amino acid arrangement and composition are highly optimized for helix stabilization in the cRev of Preparation Example 2. In the case of the N-terminal helix structure, the sequences are EDALKA and EDLLKA for /Rev and wild-type Rev protein, respectively. The sequences were confirmed to form a helix-stabilizing salt bridge between Glu− and Lys+ at positions i and i+4. The salt bridge interaction was also formed in the α-helix structure of cRev since the sequences also exist at positions i and i+4. In addition, Ala, Leu and Glu have the highest helix propensity values among amino acids. The sequence of the C-terminal helix, RQARRN, is highly interesting and provides new insight into the formation of a positively charged α-helix devoid of any negative charge necessary for salt bridge formation.

From FIG. 20E and FIG. 20F, it was confirmed that hydrogen bond interaction of Gln (Q) and Asn (N) plays an important role in the stabilization of the C-terminal helix in cRev. Therefore, based on the information descried above, it was attempted to design a novel peptide having a stable α-helix structure based on the Rev ARM of cRev and containing multiple arginine residues necessary for binding to RNA and cell penetration.

### Test Example 6. Analysis of Precise Structure of /Rev Protein and cRev Protein

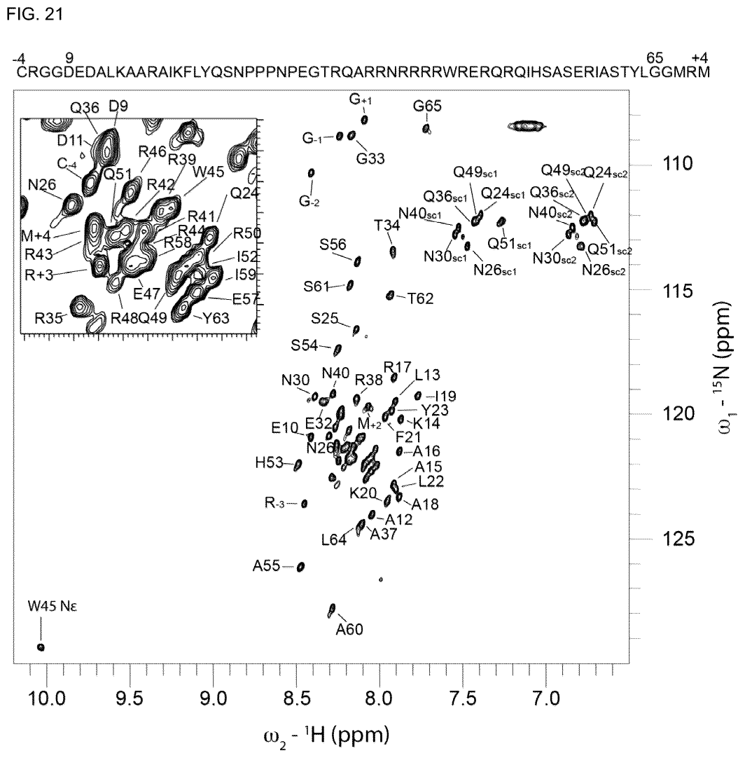

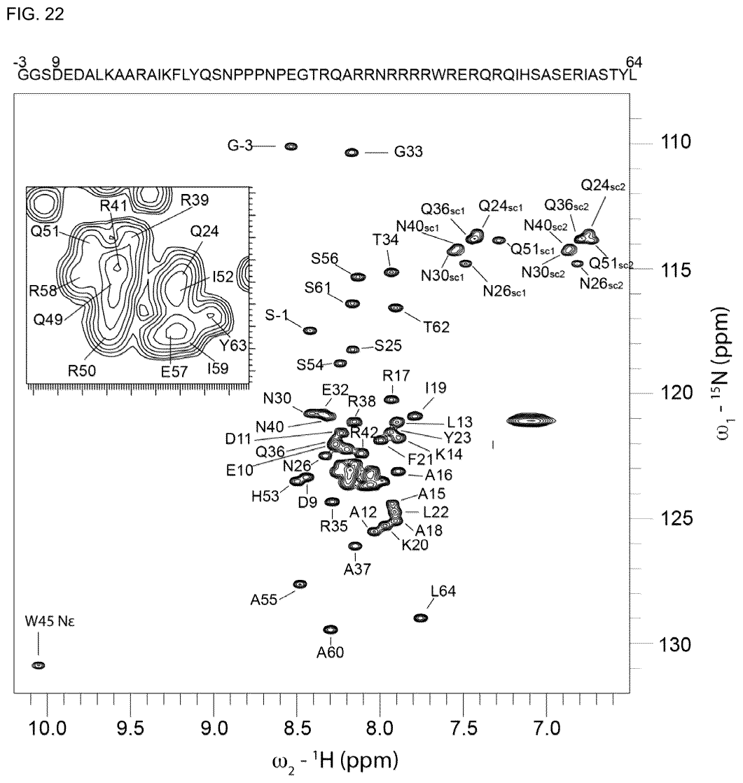

¹H-¹⁵N HSQC (heteronuclear single quantum coherence) NMR analysis was performed to more precisely compare the structure of the /Rev and cRev proteins. FIG. 21 shows the ¹H/¹⁵N HSQC spectrum of cRev and FIG. 22 shows the ¹H/¹⁵N HSQC spectrum of /Rev.

As seen from the ¹H-¹⁵N HSQC (heteronuclear single quantum coherence) spectra of FIG. 21 and FIG. 22, it can be seen that the structure of cRev is more well-resolved than that of /Rev.

### Test Example 7. Family of QARRN Motif

From the preceding test examples, it was confirmed that the region that has an arginine-rich α-helix structure and has a minimal sequence length with no negative charge in the Rev protein is the QARRN sequence (SEQ ID NO 5) of the C-terminal helix. The QARRN sequence has excellent characteristics of forming an α-helix structure while being stable within the amino acid residue sequence and having cell-penetrating ability. For the characteristics, it contains hydrogen bonds at positions I and i+4, two arginines and one helix-stabilizing alanine.

From the QARRN sequence represented by SEQ ID NO 5, QRARN, QRRAN, NRRAQ, NRARQ and NARRQ sequences of SEQ ID NOS 6-10 that satisfy all the above-mentioned conditions can be inferred through deductive reasoning. It is expected that the 5-mer sequences (i.e., QARRN motifs) will show high propensity for helix formation in other proteins, too.

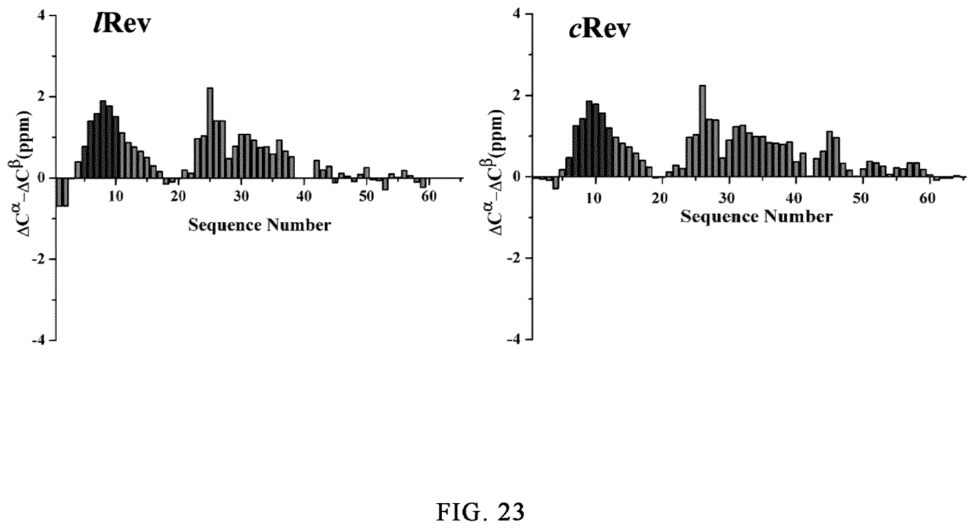

Therefore, BLAST search was performed in the PDB (Protein Data Bank) in order to identify the secondary structure of the QARRN motif in the well-known protein structures. The result is shown in FIG. 23 and Table 5. Table 5 shows the result of analyzing the occurrence of the QARRN motif in the proteomes. In Table 5, ‘the superscript^{a}’ refers to the number of cases identified from the BLAST search.

TABLE 5

| | QARRN | QRARN | QRRAN | NRRAQ | NRARQ | NARRQ |
|---|---|---|---|---|---|---|
| α-Helix | 3^{a} | 0 | 1 | 3 | 5 | 7 |
| β-Sheet | 0 | 0 | 1 | 1 | 1 | 0 |
| Coil | 0 | 0 | 0 | 0 | 0 | 0 |
| Helix | 100% | N/A | 50% | 75% | 83% | 100% |
| percentage | | | | | | |

FIG. 23 shows the result of analyzing the chemical shift index related to secondary structure using the NMR data of cRev and/Rev. A region having an α-helix structure in cRev (shown in red) was confirmed.

Through the above-described analysis, it was confirmed that the sequence in the Rev protein sequence that has the characteristics of α-structure stabilization, cell-penetrating ability, minimum sequence length, sequence structure with no negative charge and specific binding ability to RNA is QARRN represented by SEQ ID NO 5. Based on this basic structure, sequences represented by SEQ ID NOS 6-10, which are expected to have the same effect as that of sequence of SEQ ID NO 5, were inferred through deductive reasoning.

### Test Example 8. Analysis of Recognition Mechanism of Rev-RRE

The RRE RNA recognition mechanism of the various conformers of the Rev protein was analyzed and a series of processes that occur during the initial phase for the conformers of the Rev protein were inferred and shown schematically.

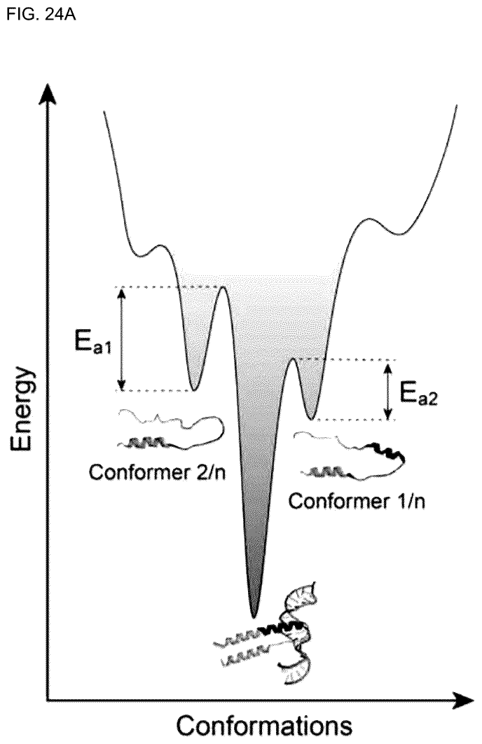

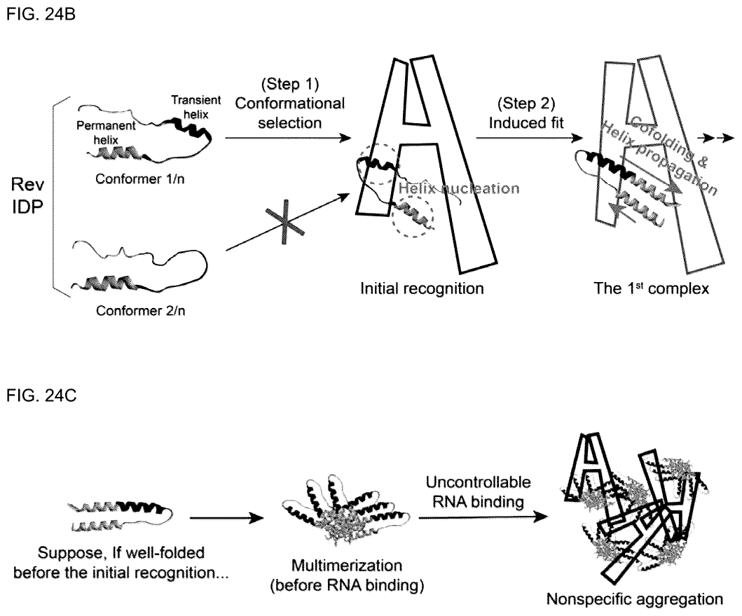

FIGS. 24A to 24C show the Rev-RRE recognition model which schematically shows how the intrinsic disorder of Rev affects multimerization and specificity. FIG. 24A shows the energy landscape of the Rev conformer (Eₐ₁>Eₐ₂), FIG. 24B shows the initial process of the Rev-RRE recognition model, and FIG. 24C shows the result of analyzing the raison d'etre of the intrinsic disorder in Rev.

As shown in FIG. 24A, it is expected that the presence of the N-terminal helix and the C-terminal helix in the Rev protein will exert a major effect in the Rev-RRE recognition step. This is a new mechanistic insight distinguished from the numerous previous studies.

In general, according to the properties of the conventional Rev conformers, it is obvious that the potential energy of general Rev conformers or a conformer in which the N-terminal helix is stabilized (conformer 2/n) will be higher than the potential energy of a conformer in which both helices are stabilized (conformer 1/n) (that is to say, the conformer 1/n has lower potential energy than the conformer 2/n) (FIG. 24A).

The C-terminal helix of the conformer in which both helix structures are stabilized (conformer 1/n) is selected first in the initial recognition step via conformational selection mechanism because it is complementary in shape to the IIB site of RRE (FIG. 24B). After the initial interaction with RNA, the helices at the N-terminal and the C-terminal participate in nucleation, which is accompanied by the structural change of Rev to form the OLIGO domain, resulting in the completion of the HLH motif structure. In other words, this process follows the induced fit mechanism. After the first complex formation of Rev and RRE RNA is completed, protein-protein interaction and additional protein-RNA interaction are formed at the hydrophobic residue of the HLH motif, which leads to further Rev multimerization of the Rev protein. The mechanism involving conformational selection followed by induced fit contributes not only to enhancing the selectivity but also to preventing the uncontrollable multimerization of Rev prior to RRE binding.

### Test Example 9. Structural Analysis of Cell-Penetrating Peptides (CPPα)

In general, the cell membrane is negatively charged. Therefore, the absence of any negatively charged residue is important in achieving high cell penetration efficiency because it minimizes the electrostatic repulsion between the CPP and the negatively charged cell membrane. Accordingly, cell-penetrating peptides with various sequence structures (Examples 1-14; CPP1 to CPP13 and CPP11d) were prepared and their structures were analyzed to check whether they can easily penetrate cells.

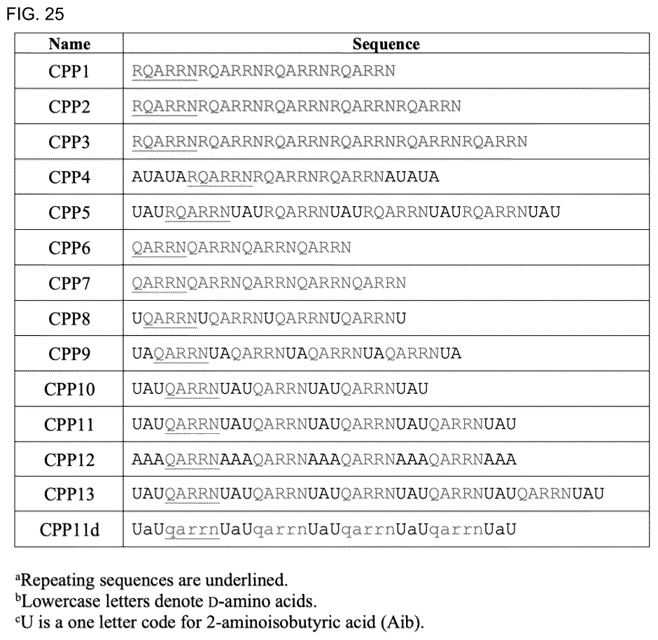

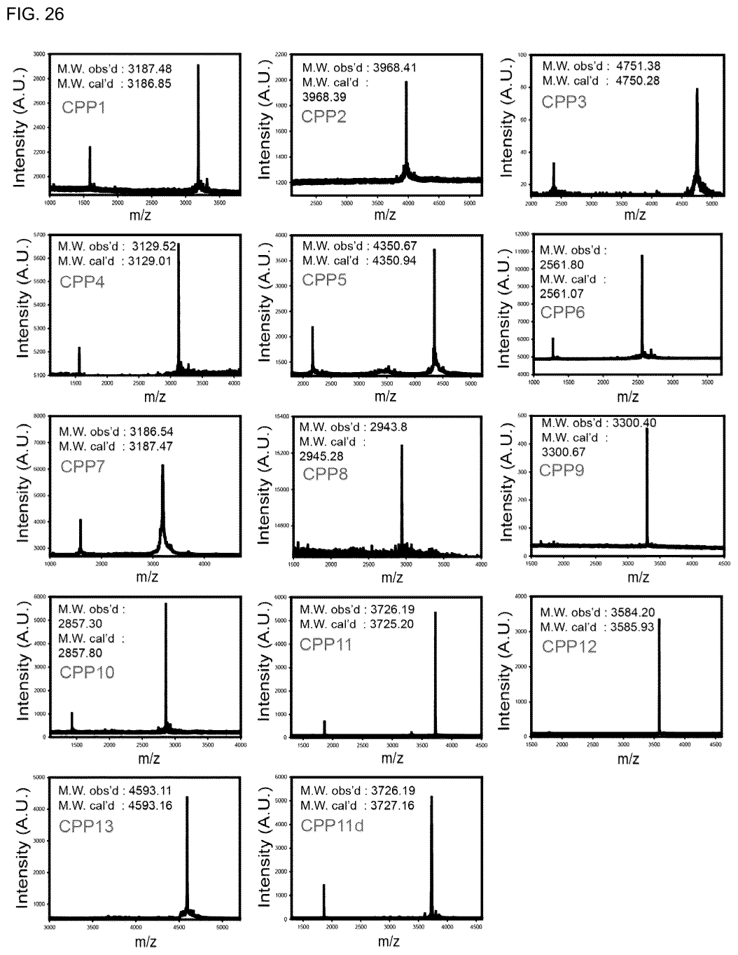

FIG. 25 shows the sequences of the cell-penetrating peptides represented by SEQ ID NOS 15-24 (CPP5 to CPP13 and CPP11d). The lowercase letters stand for D-amino acids and U stands for Aib (2-aminoisobutyric acid). FIG. 26 shows the MALDI-TOF MS spectra of the cell-penetrating peptides prepared in Examples 1-14 (CPP1 to CPP13 and CPP11d).

As shown in FIG. 25 and FIG. 26, the QARRN motif (SEQ ID NO 5) according to the present disclosure does not form a stable helix structure by itself. However, since the QARRN motif of SEQ ID NO 5 is arginine-rich and has a cell-penetrating α-helix (CPPα) structure without any negatively charged residue, the cell-penetrating peptides of Examples 1-14 were designed based thereon. Specifically, in anticipation of the increase in helix stability through back-and-forth helix nucleation and propagation, cell-penetrating peptides containing tandem repeats of QARRN sequences were designed. As shown in FIG. 26, the peptides were synthesized successfully.

CD analysis was performed on the 14 cell-penetrating peptides (CPPα) prepared in Examples 1-14. The cell-penetrating peptides (CPP1 TO 13 and CPP11d) were dissolved respectively in 10 mM potassium phosphate. The analysis was performed under the condition of pH 7.4 and 20° C. In the analysis result, MRE stands for mean residue ellipticity.

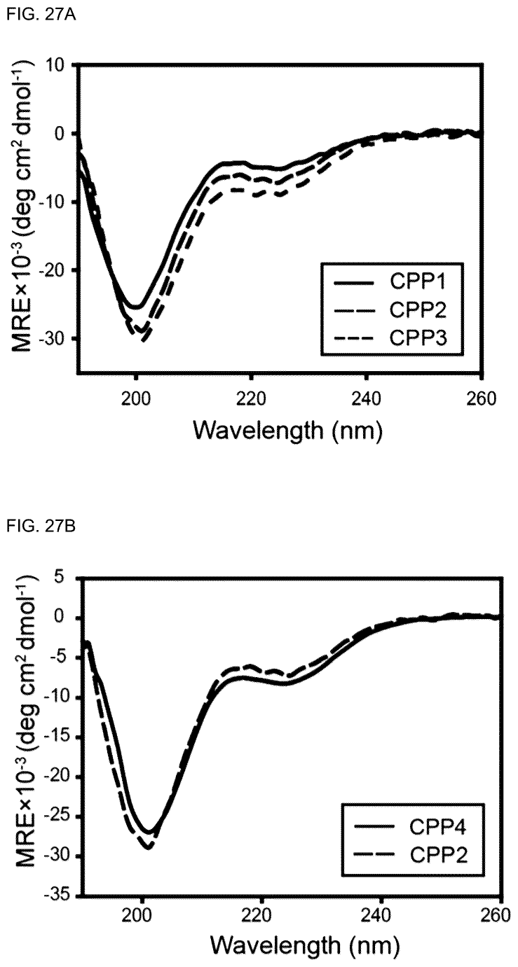

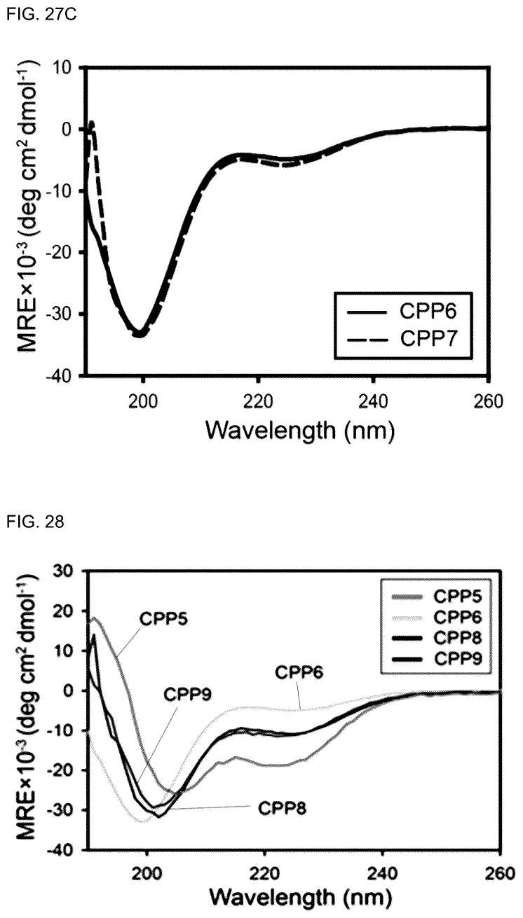

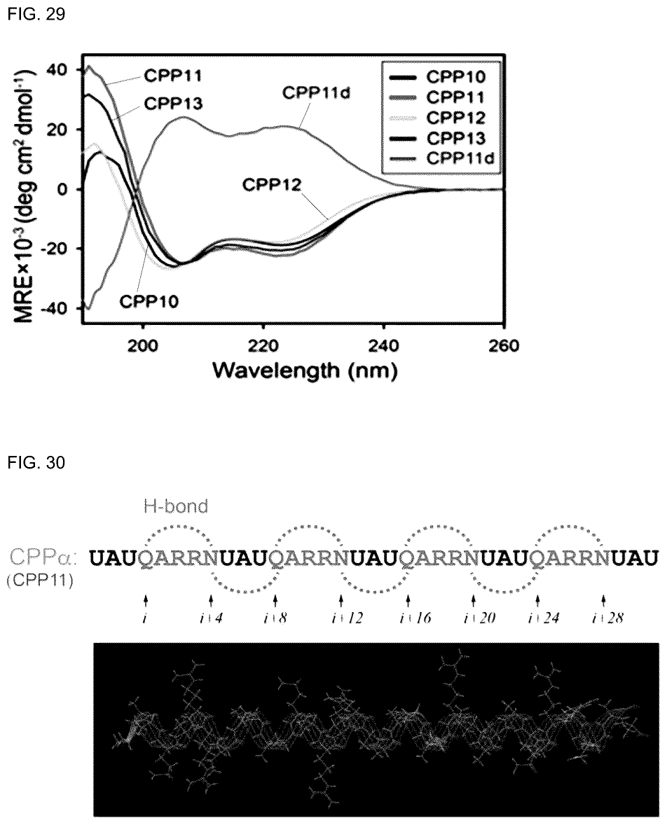

FIG. 27A to 27C shows the CD spectra of the 6 cell-penetrating peptides prepared in Examples 1-4 and 6 and 7 (CPPα). FIG. 28 and FIG. 29 show the CD spectra of the 9 cell-penetrating peptides prepared in Examples 5 and 6 and 8-14 (CPPα).

The cell-penetrating peptides of Examples 1-3, 6 and 7 (CPP1, CPP2, CPP3, CPP6 and CPP7) were expected to maintain stabilized helical structure due to propagation of the back-and-forth helix structure. However, as shown in FIG. 27A and FIG. 27C, it was confirmed that they exist mostly in random coil state where the helical structures are not highly stabilized.

Based on the results described above, cell-penetrating peptides (CPP4 and CPP5; Examples 4 and 5) were prepared by adding intervening amino acids with high helix propensity (P_{α}>1.0), such as Ala (A) and Aib (U), between the RQARRN repeating sequence. As a result (FIG. 27B and FIG. 28), it was confirmed that the helical structure was somewhat stabilized. For amino acids with high helix propensity, refer to Chou and Fasman, Biochemistry, 1974, 13, 211-222.

Next, the effect of increasing number of intervening amino acids was analyzed. As shown in FIG. 28 and FIG. 29, the helicity of the cell-penetrating peptide of Example 10 (CPP10 (27-mer)) was significantly higher than those of CPP8 and CPP9. Through this, it can be seen that, while the presence of one or two intervening amino acid has little effect on the stabilization of the helical structure, a significant effect of stabilization of the helical structure can be achieved when there exist 3 intervening amino acids.

In addition, as shown in FIG. 29, the cell-penetrating peptide of Example 11 (CPP11 (35-mer)) has a perfect α-helical structure. Therefore, it can be seen that it is the most desirable that the QARRN repeating sequence is repeated 4 or more times.

In addition, as shown in FIG. 29, the result of comparing the helicity of the cell-penetrating peptide of Example 11 (CPP11) and the cell-penetrating peptide of Example 12 (CPP12) reveals that UAU is advantageous over AAA as the intervening sequence in terms of stability.

In addition, as shown in FIG. 29, the degree of stabilization of the α-helical structure is not further increased for the cell-penetrating peptide of Example 13 (CPP13 (43-mer)), indicating the stabilization of the α-helical structure is not increased further when the number of the QARRN tandem repeat exceeds 5.

As shown in FIG. 29, it was confirmed that the CPP11d of Example 14 wherein QARRN was substituted with D-amino acids has a mirror image CD profile for CPP11. This shows that the CPP11d has a perfect left-handed helical conformation.

### Test Example 10. Evaluation of Thermal Stability of Cell-Penetrating Peptides (CPPα)

For the cell-penetrating peptide prepared in Example 11 (CPP11), CD spectra were measured under various temperature conditions (4° C., 20° C., 36° C., 52° C., 68° C. and 84° C.) to analyze thermal stability through equilibrium thermal unfolding.

In addition, the CD spectra of the cell-penetrating peptide prepared in Example 11 (CPP11) at various concentrations (5-200 μM) were measured at 20° C., and molar ellipticity ratio at 222 nm and 208 nm ([θ]₂₂₂/[θ]₂₀₈) was calculated and analyzed.

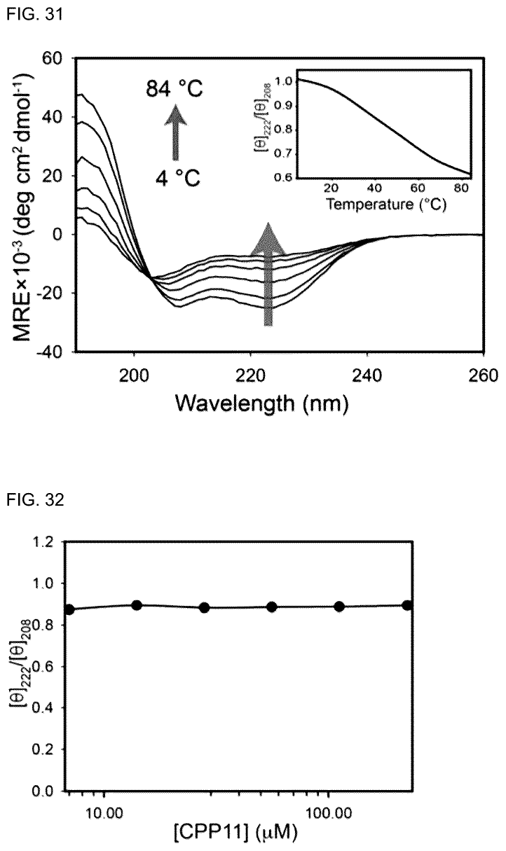

FIG. 30 schematically shows the hydrogen bonding of the cell-penetrating peptide prepared in Example 11 (CPP11). FIG. 31 shows the CD spectra of the cell-penetrating peptide prepared in Example 11 (CPP11) at different temperature conditions (4° C., 20° C., 36° C., 52° C., 68° C. and 84° C.). FIG. 32 shows the CD spectra ([θ]₂₂₂/[θ]₂₀₈) of the cell-penetrating peptide prepared in Example 11 (CPP11) depending on concentration.

As shown in FIG. 30, it was found that all glutamine (Q) and asparagine (N) residues exist separately at positions i and i+4 in the cell-penetrating peptides according to the present disclosure (CPP10, CPP11, CPP12, CPP13 and CPP11d) and they play a major role in stabilizing the α-helical structure through formation of hydrogen bonding.

As shown in FIG. 31, it can be seen from the distinct isodichroic point at 203 nm that the cell-penetrating peptide prepared in Example 11 (CPP11) undergoes two-state transition between the coil conformation and helical conformation depending on temperature. The cell-penetrating peptide prepared in Example 11 (CPP11) had a melting temperature of 39.4° C., indicating that the cell-penetrating peptide of the present disclosure can stably maintain its superior α-helical structure at human body temperature (˜37° C.).

As shown in FIG. 32, the cell-penetrating peptide prepared in Example 11 (CPP11) was not affected by concentration because it exists as monomers in solution.

### Test Example 11. Comparison of Structure of Cell-Penetrating Peptide (CPPα) with Tat

The cell-penetrating peptide according to the present disclosure has a unique secondary structure, which is predicted as a result of structural difference from the existing cell-penetrating peptides. To confirm this, CD analysis was performed on the disordered Tat peptide derived from HIV-1 and the cell-penetrating peptide prepared in Example 11 (CPP11). The HIV-1-derived disordered Tat peptide (47-57; Tyr-Gly-Arg-Lys-Lys-Arg-Arg-Gln-Arg-Arg-Arg; SEQ ID NO 41) was synthesized in the laboratory and used as a comparative example.

FIG. 33 shows the CD spectra of the disordered Tat peptide (Comparative Example) derived from HIV-1 and the cell-penetrating peptide prepared in Example 11 (CPP11). It can be seen that the cell-penetrating peptide according to the present disclosure (CPP11) shows distinct structural difference from Tat, which is a representative cell-penetrating peptide.

### Test Example 12. Analysis of Cellular Uptake Efficiency of Cell-Penetrating Peptides (CPPα)

HeLa, HCT116 and 293T cells were respectively treated with 30 μM of the cell-penetrating peptide prepared in Example 10 (CPP10), the cell-penetrating peptide prepared in Example 11 (CPP11) or an existing cell-penetrating peptide (Tat, SEQ ID NO 41), respectively, and flow cytometry was performed to measure cellular uptake efficiency. The cell-penetrating peptides (CPP10, CPP11 and Tat) were labeled with FAM (5 (6)-carboxyfluorescein) at the N-terminal. MFI stands for mean fluorescence intensity. Untreated cells were used as control groups (cell only). The cellular uptake efficiency refers to the degree of penetration into cells.

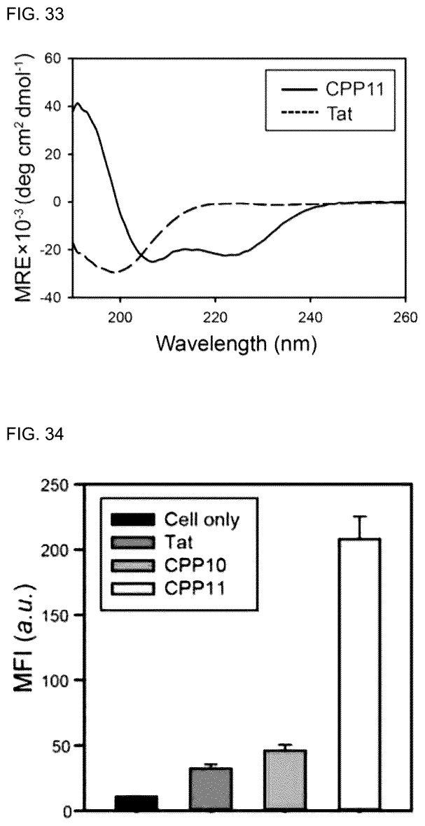

FIG. 34 shows the flow cytometry result for the Hela cells treated with the cell-penetrating peptide prepared in Example 10 (CPP10), the cell-penetrating peptide prepared in Example 11 (CPP11) or the existing cell-penetrating peptide (Tat) (n=3) (mean±S.D.).

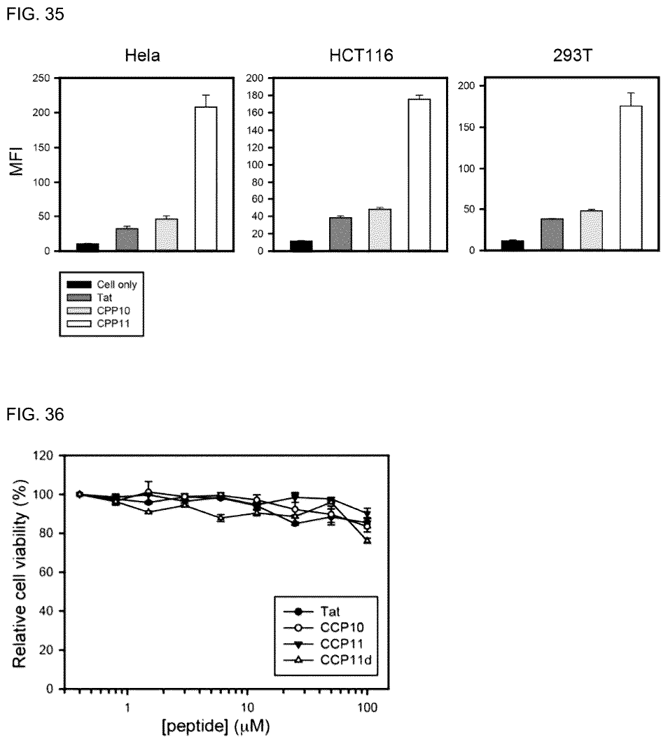

FIG. 35 shows the flow cytometry result for the Hela cells, HCT116 cells and 293T cells treated with the cell-penetrating peptide prepared in Example 10 (CPP10), the cell-penetrating peptide prepared in Example 11 (CPP11) or the existing cell-penetrating peptide (Tat) (n=3) (mean±S.D.).

As a result of the flow cytometry, it was confirmed that the cell-penetrating peptide prepared in Example 10 (CPP10) has significantly superior cell-penetrating ability than the existing cell-penetrating peptide (Tat), as shown in FIG. 34 and FIG. 35.

The cell-penetrating peptide prepared in Example 11 (CPP11) (CPPα) had significantly remarkable cellular uptake efficiency of 4 times or more than that of the Tat peptide (Tat). This tendency was observed in both the cancer cells (HeLa and HCT116 cells) and non-cancerous cells (293T cells). That is to say, it can be seen that the cell-penetrating peptide prepared in Example 11 (CPP11) has superior cell-penetrating ability of 4 times or more than that of Tat regardless of cell type.

### Test Example 13. Analysis of Cytotoxicity of Cell-Penetrating Peptides (CPPα)

Cytotoxicity analysis was performed on the cell-penetrating peptide prepared in Example 10 (CPP10), the cell-penetrating peptide prepared in Example 11 (CPP11) and the cell-penetrating peptide prepared in Example 14 (CPP11d).

FIG. 36 shows a result of treating Hela cells with the cell-penetrating peptide prepared in Example 10 (CPP10), the cell-penetrating peptide prepared in Example 11 (CPP11) or the cell-penetrating peptide prepared in Example 14 (CPP11d) and measuring cytotoxicity with a WST-8 reagent.

As shown in FIG. 36, it was confirmed that the cell-penetrating peptide prepared in Example 10 (CPP10), the cell-penetrating peptide prepared in Example 11 (CPP11) and the cell-penetrating peptide prepared in Example 14 (CPP11d) are safe substances that hardly show cytotoxicity.

### Test Example 14. Analysis of Cell-Penetrating Ability of Cell-Penetrating Peptide (CPPα) at Different Concentrations

Flow cytometry was performed after treating Hela cells with the cell-penetrating peptide prepared in Example 11 (CPP11) of different concentrations (4 μM, 30 μM, 60 μM, 90 μM, 120 μM, 270 μM and 500 μM).

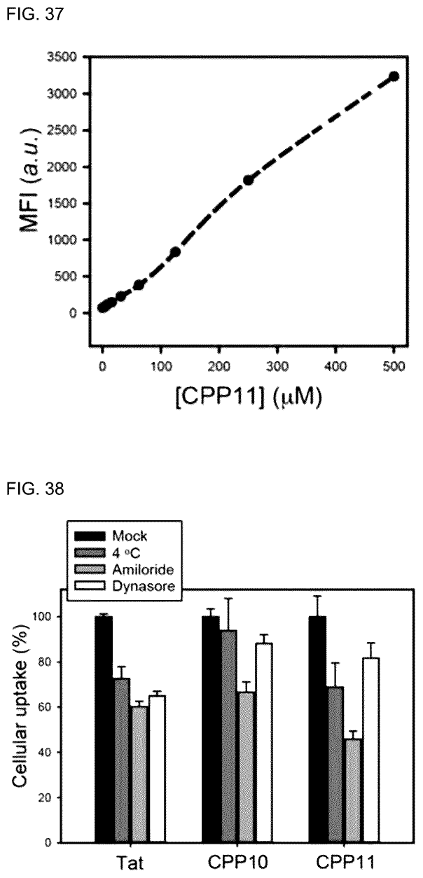

FIG. 37 shows the flow cytometry result for the Hela cells treated with the cell-penetrating peptide prepared in Example 11 (CPP11) at different concentrations.

As shown in FIG. 37, MFI increases as the concentration of the cell-penetrating peptide prepared in Example 11 (CPP11) increases, indicating that uptake efficiency increases gradually with the concentration.

### Test Example 15. Analysis of Cell Penetration Pathway of Cell-Penetrating Peptides (CPPα)

The cellular uptake/penetration pathway of the cell-penetrating peptide according to the present disclosure was investigated. Specifically, the cell penetration pathway of the cell-penetrating peptide prepared in Example 10 (CPP10) and the cell-penetrating peptide prepared in Example 11 (CPP11) was investigated by treating with endocytosis inhibitors. For inhibition of endocytosis, experiment was performed at low temperature using amiloride and dynasore as the inhibitors. Amiloride is a macropinocytosis inhibitor and dynasore is an inhibitor that inhibits endocytoses mediated by clathrin and caveolae. 10 μM of the cell-penetrating peptide prepared in Example 10 (CPP10) or the cell-penetrating peptide prepared in Example 11 (CPP11) labeled with FAM was used for flow cytometry. The Tat peptide was used under the same condition as a comparative example.

FIG. 38 shows the result of treating Hela cells with the cell-penetrating peptide prepared in Example 10 (CPP10), the cell-penetrating peptide prepared in Example 11 (CPP11) or the Tat peptide and measuring cellular uptake thereof by flow cytometry (n=3) (mean±S.D.).

As shown in FIG. 38, the cellular uptake efficiency of all the three CPPs (Tat, CPP10 and CPP11) was decreased slightly at low temperature. It was confirmed that the cell-penetrating peptides are partially involved in energy-dependent endocytosis in addition to direct penetration mechanism.

Among the three cell-penetrating peptides, CPP10 and CPP11 having a helical structure were inhibited to a greater extent for amiloride than dynasore. That is to say, CPP10 and CPP11 according to the present disclosure penetrate into cells mainly via macropinocytosis. In summary, it can be seen that CPP10 and CPP11, which are the helical cell-penetrating peptides according to the present disclosure, penetrate into cells via both energy-independent direct penetration and energy-dependent endocytosis.

### Test Example 16. Analysis of Cellular Localization of Cell-Penetrating Peptide (CPPα)

Hela cells were treated with 10 μM of the cell-penetrating peptide prepared in Example 11 (CPP11) or the Tat peptide, cultured for 2 hours and then observed by CLSM (Confocal laser scanning microscopy). The cell-penetrating peptides were labeled with FAM.

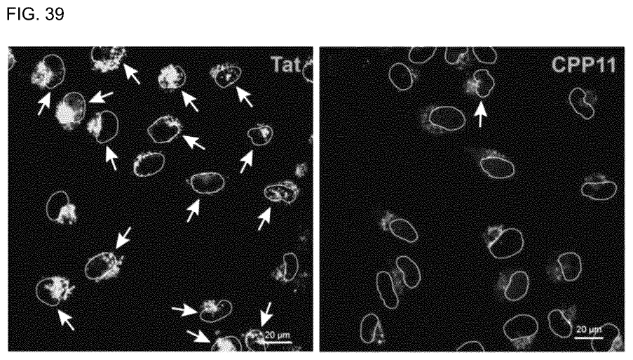

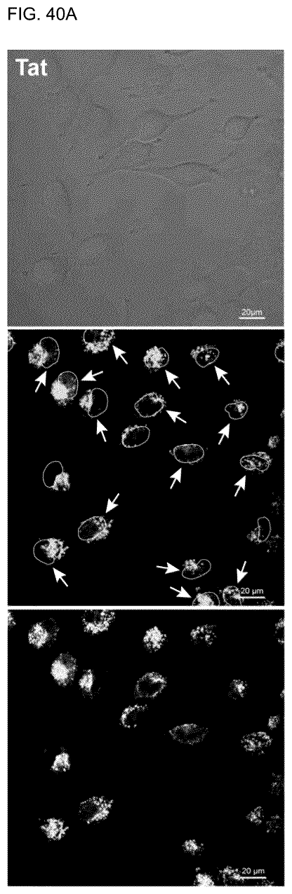

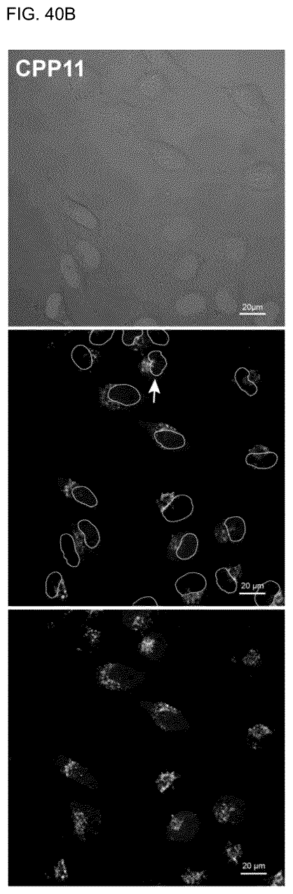

FIG. 39 shows the CLSM (confocal laser scanning microscopy) images of the Hela cells treated with the cell-penetrating peptide prepared in Example 11 (CPP11) or the Tat peptide. The yellow lines indicate cell nuclei stained with DAPI.

FIGS. 40A and 40B show the CLSM (confocal laser scanning microscopy) images of the Hela cells treated with the cell-penetrating peptide prepared in Example 11 (CPP11) or the Tat peptide, comparing cellular localization behavior more specifically. The yellow lines indicate cell nuclei stained with DAPI.

As shown in FIG. 39 and FIGS. 40A to 40B, the location of the cell-penetrating peptides within the cells was identified by confocal laser scanning microscopy (CLSM). Whereas the cell-penetrating peptide Tat is located mainly in the nucleolus, the cell-penetrating peptide of Example 11 (CPP11) according to the present disclosure is mainly located outside the nucleus. It is thought that the different locations of the Tat and CPP11 within cells is due to structural differences (Tat is in the form of a random coil, while CPP11 has a perfect α-helical structure).

The nucleolus is a membraneless organelle or condensate formed by LLPS (liquid-liquid phase separation). One of the main driving forces of LLPS is the weak, multivalent electrostatic interaction between intrinsically disordered biomolecules with low complexity. It is known that RNA-binding proteins are abundant in intrinsically disordered regions and low-complexity sequences compared to the entire proteome. The 13-mer CPP Tat (Tat48-60) is derived from the RNA-binding domain of the HIV-1 Tat protein. Most of the existing cell-penetrating peptides such as Tat have intrinsically disordered structures such as the random coil structure, are positively changed and have low complexity. That is to say, disordered Tat would accumulate in the nucleolus together with nucleolar RNA through LLPS.

On the other hand, the cell-penetrating peptide according to the present disclosure has a perfect α-helical structure with low conformational freedom unlike the existing cell-penetrating peptides such as Tat, and is advantageous in that it tends not to bind to RNA through nonspecific electrostatic interaction.

### Test Example 17. Analysis of Cellular Localization of Cell-Penetrating Peptide (CPPα)

The EMSA of yeast tRNA or IIB RRE was analyzed for the cell-penetrating peptide prepared in Example 11 (CPP11) or the Tat peptide. Afterwards, EMSA (electrophoretic mobility shift assay) was conducted on agarose gel. From the EMSA, R_{f} (retention factor) was calculated by the following equation.

$${R}_{f} = 1 - y / x ( see FIG . 41 ) \qquad [ Equation 1 ]$$

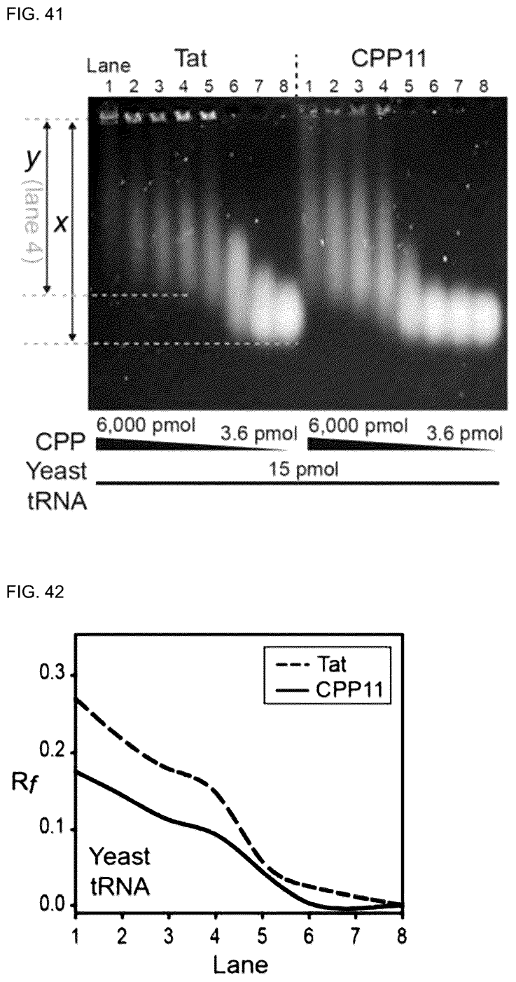

FIG. 41 shows the EMSA (electrophoretic mobility shift assay) images of yeast tRNA for the cell-penetrating peptide prepared in Example 11 (CPP11) or the Tat peptide. FIG. 42 shows a result of quantifying R_{f} (retention factor) from the EMSA result of FIG. 41.

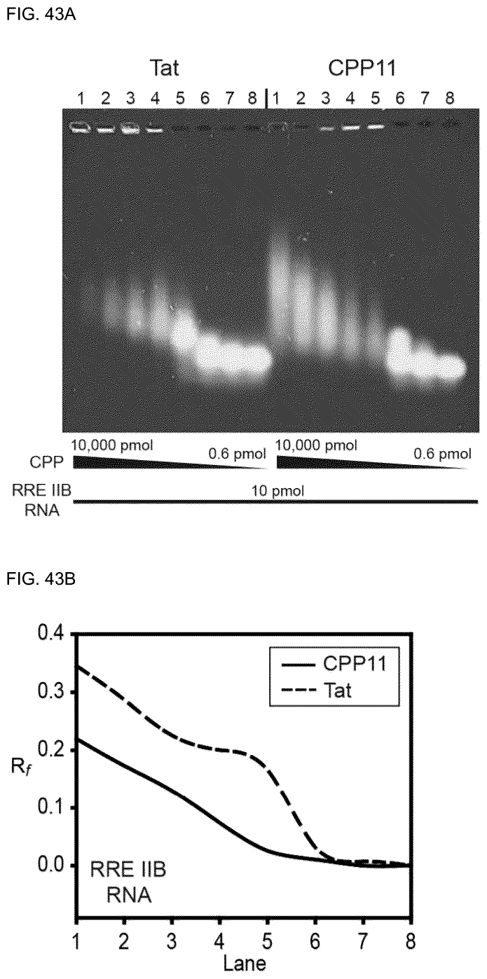

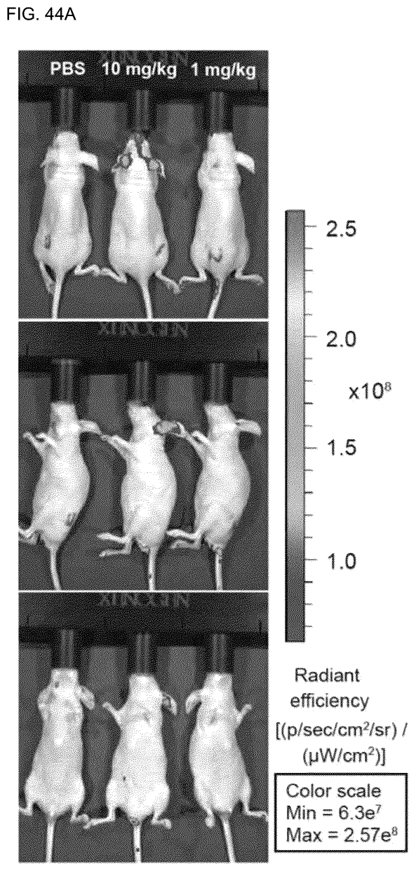

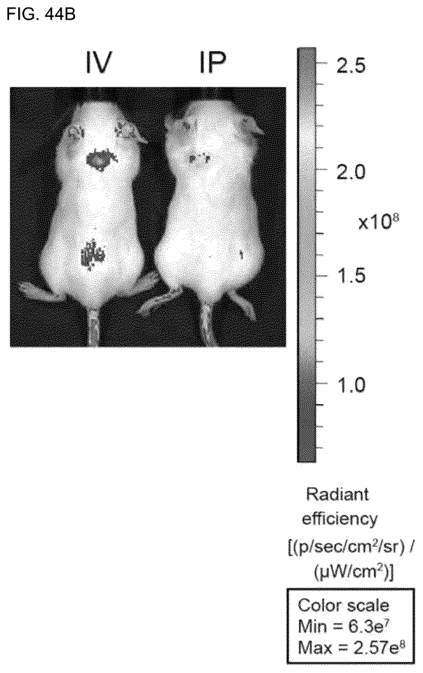

FIGS. 43A and 43B show a result of analyzing the nonspecific electrostatic interaction between the cell-penetrating peptide prepared in Example 11 (CPP11) or the Tat peptide and heterologous RNA (IIB RNA). FIG. 43A shows the EMSA analysis result for the cell-penetrating peptide prepared in Example 11 (CPP11) or the Tat peptide, and FIG. 43B shows a result of quantifying Rr from FIG. 43A.

The degree of nonspecific electrostatic interaction with a non-cognate RNA model was investigated using EMSA (electrophoretic mobility shift assay). As a result, it was found out that Tat has qualitatively and quantitatively higher affinity for RNA than CPP11. That is to say, it can be seen that the existing cell-penetrating peptides such as Tat forms nonspecific interaction with RNA.

As shown in FIGS. 43A and 43B, the EMSA result in the presence of IIB RNA shows that Tat has qualitatively and quantitatively higher affinity for RNA than CPP11.

In summary, it can be seen that the cell-penetrating peptide according to the present disclosure, which has a stable rod-like α-helical structure, exhibits a cellular localization behavior distinguished from that of other disordered arginine-rich CPPs.

### Test Example 17. Analysis of In-Vivo Targeting Characteristics of Cell-Penetrating Peptides (CPPα)

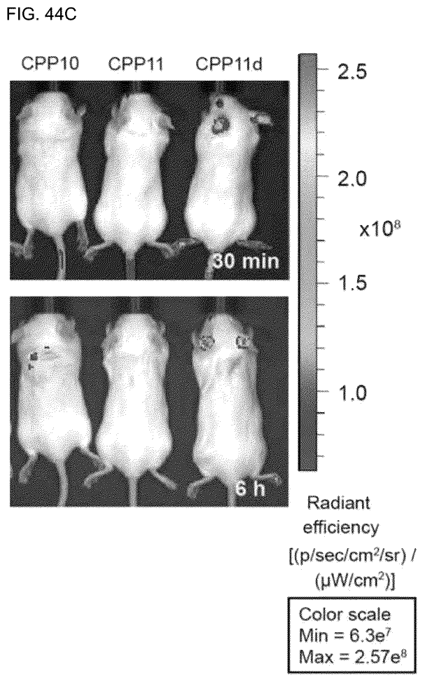

Female 7-week-old BALB/c nude mice were injected IV (intravenously) with the cell-penetrating peptide prepared in Example 10 (CPP10) at 10 mg/kg or 1 mg/kg via the lateral tail vein and imaged using IVIS (in-vivo imaging system) (PerkinElmer, USA) 4 hours later. The images were analyzed with the Living Image software (PerkinElmer). The mice were analyzed from the top, bottom (belly) and side positions (FIG. 44A). Then, after administering the same volume of the cell-penetrating peptide prepared in Example 10 (CPP10) at 10 mg/kg via IV (intravenous) or IP (intraperitoneal) injection, the mice were imaged 4 hours later (FIG. 44B).

Next, female 7-week-old BALB/c nude mice were injected IV (intravenously) with the cell-penetrating peptide of Example 10 (CPP10), the cell-penetrating peptide of Example 11 (CPP11) or the cell-penetrating peptide of Example 14 (CPP11d) at 10 mg/kg via the lateral tail vein, and they were imaged 30 minutes and 6 hours later using IVIS (in-vivo imaging system) (PerkinElmer, USA). The images were analyzed with the Living Image software (PerkinElmer) (FIG. 44C). In FIGS. 44A and 44B, the cell-penetrating peptides were labeled with TAMRA (5 (6)-carboxytetramethylrhodamine). In FIG. 44C, the cell-penetrating peptides were labeled with FAM.

FIG. 44A shows the in-vivo fluorescence images of the mice treated with the cell-penetrating peptide of Example 10 (CPP10). FIG. 44B shows the in-vivo fluorescence image of the mice to which the cell-penetrating peptide of Example 10 (CPP10) was administered by IP (intraperitoneal) injection and IV injection. FIG. 44C shows the in-vivo fluorescence images of the mice to which the cell-penetrating peptide of Example 10 (CPP10), the cell-penetrating peptide of Example 11 (CPP11) or the cell-penetrating peptide of Example 14 (CPP11d) was administered by IV injection.

When the cell-penetrating peptide of Example 10 (CPP10) fluorescence-labeled with TAMRA was administered via the tail vein, bright fluorescence was observed in the ear of the mice (FIG. 44A).

As shown in FIG. 44B, the same volume of the cell-penetrating peptide of Example 10 (CPP10) showed higher targeting efficiency when administered by IV injection rather than by IP injection.

As shown in FIG. 44C, all the cell-penetrating peptides according to the present disclosure (CPP10, CPP11 and CPP11d) targeted the ear. Among them, the accumulation efficiency of the CPP11d prepared in Example 14 in the ear was much higher than those of the other two cell-penetrating peptides.

Since there was no difference depending on the fluorescence labeling of the cell-penetrating peptides with TAMRA or FAM, it can be seen that TAMRA or FAM does not affect the cell penetration of the cell-penetrating peptides or their pathways. That is to say, it was confirmed that the cell-penetrating ability of the cell-penetrating peptide according to the present disclosure is not lost or decreased due to binding with a fluorophore or a drug, and it can successfully deliver them into the target site in the body.

It was confirmed that the cell-penetrating peptide of Example 14 (CPP11d) has higher accumulation and retention efficiency because it is highly resistant to proteolytic enzymes.

Most of the existing CPPs target the liver, kidney, lung, brain, heart, spleen, lymphocytes, tumors, etc. The cell-penetrating peptide according to the present disclosure has a remarkably superior effect in that it targets the ear unlike the existing cell-penetrating peptides.

### Test Example 18. Analysis of In-Vivo Targeting Characteristics of Cell-Penetrating Peptide (CPP11)

Female 7-week-old BALB/c nude mice were injected IV (intravenously) with the cell-penetrating peptide prepared in Example 11 (CPP11) or a buffer solution (PBS; w/o treatment) at 10 mg/kg via the lateral tail vein, and the experimental animals were anesthetized and euthanized 4 hours later. After extracting major organs (ear, brain, heart, lung, liver, spleen and kidney) from the euthanized experimental animals, the tissues were imaged using IVIS (in-vivo imaging system) (PerkinElmer, USA) and the images were analyzed with the Living Image software (PerkinElmer).

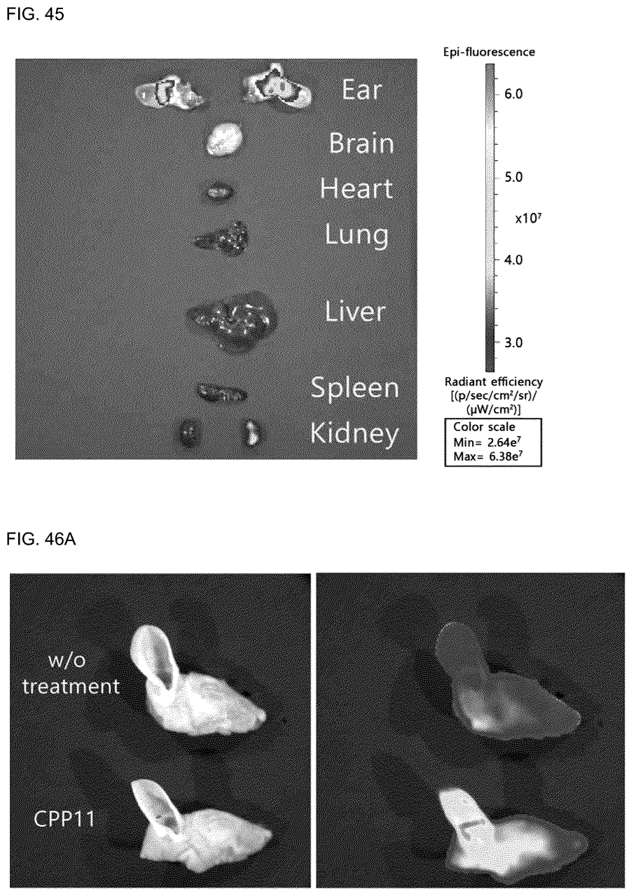

FIG. 45 shows the IVIS images of the major organs extracted 4 hours after the injection of the cell-penetrating peptide prepared in Example 11 (CPP11) via the tail vein.

As shown in FIG. 45, it was confirmed that the cell-penetrating peptide according to the present disclosure (CPP11) is not accumulated in the heart, lung and spleen tissues but targets mainly the ear even when administered via the tail vein (the accumulation rate in the brain was very low). It was confirmed that the CPP11 prepared in Example 11 is accumulated in the ear within 4 hours and is excreted out of the body through the liver and kidneys.

FIGS. 46A and 46B show the IVIS images of the outside (FIG. 46A) and inside (FIG. 46B) of the head (including the ear) 4 hours after the injection of the cell-penetrating peptide prepared in Example 11 (CPP11) or the buffer solution (PBS; w/o treatment) via the tail vein.

As shown in FIGS. 46A and 46B, it was confirmed that the cell-penetrating peptide of the present disclosure (CPP11), having a sequence composed of the 5-mer QARRN (SEQ ID NO 5) repeating unit and UAU (linker), was accumulated effectively in the facial area, especially in the ear, in large quantities even though it was administered via the tail vein. The cell-penetrating peptide (CPP11) was not administered for the w/o treatment, which was used as a control group to confirm and remove background signals.

From FIG. 46B, it can be seen that the cell-penetrating peptide of the present disclosure (CPP11) was accumulated deeply not only in the ear but also in the nose.

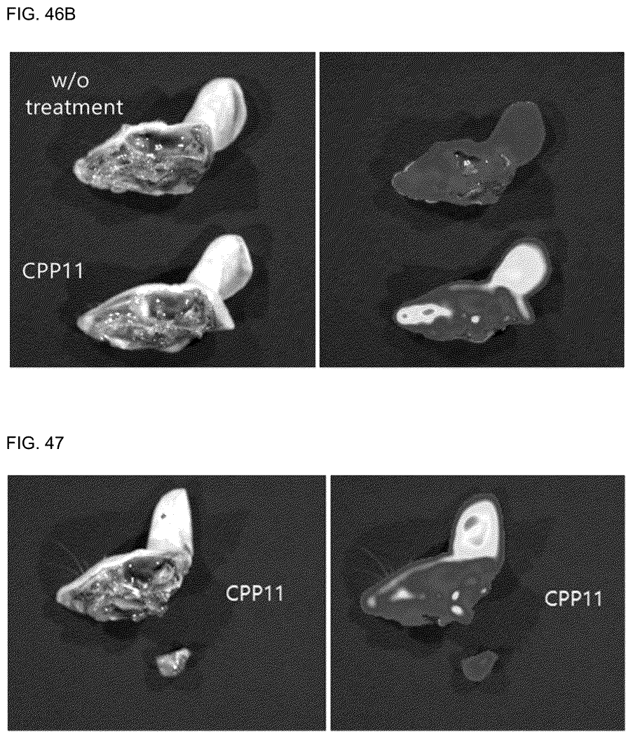

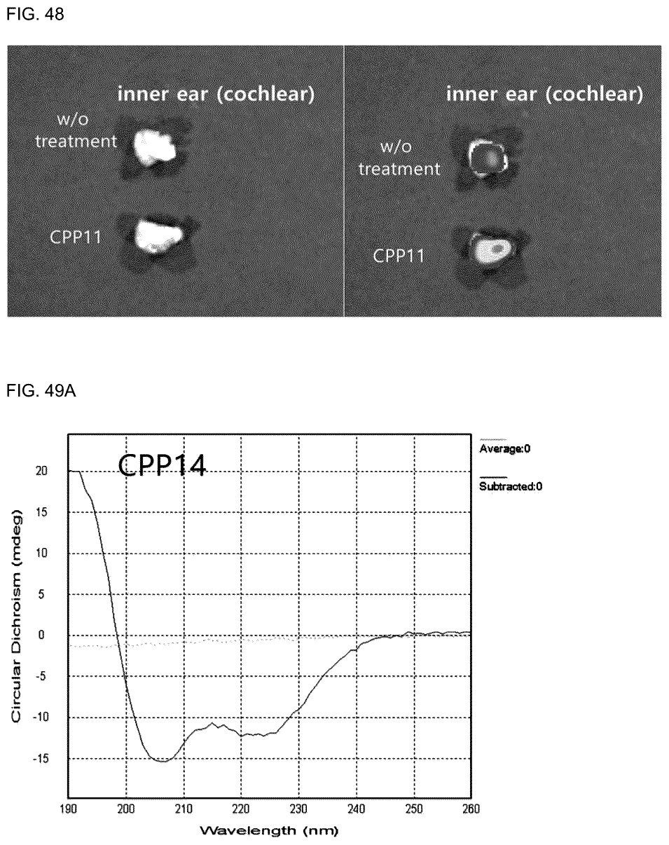

FIG. 47 shows the IVIS images of the cochlea (inner ear) separated from the head and the ear 4 hours after the injection of the cell-penetrating peptide prepared in Example 11 (CPP11) via the tail vein. FIG. 48 shows the IVIS images of the cochlea (inner ear) separated from the ear of the experimental animal after administration of the cell-penetrating peptide prepared in Example 11 (CPP11) or treatment with the buffer solution (PBS; w/o treatment).

As shown in FIG. 47, the cell-penetrating peptide of the present disclosure (CPP11), having a sequence composed of the 5-mer QARRN (SEQ ID NO 5) repeating unit and UAU (linker), was accumulated effectively in the facial area, especially in the ear, in large quantities even though it was administered via the tail vein. The small tissue shown at the bottom is the inner ear (cochlea) separated from the ear.

As shown in FIG. 48, significantly remarkable accumulation in the inner ear (cochlea) was confirmed when the cell-penetrating peptide of the present disclosure (CPP11) was administered as compared to the w/o treatment (control group) wherein the cell-penetrating peptide (CPP11) was not administered.

### Test Example 19. Analysis of α-Helix Structure of Cell-Penetrating Peptides (CPP14 to 18)

The structure of the cell-penetrating peptides prepared in Examples 15-19 (CPP14 to CPP18) was analyzed to confirm whether they can easily penetrate cells. The cell-penetrating peptides of Example 15-19 are cell-penetrating peptide variants prepared by changing the sequence of the 5-mer repeating unit from SEQ ID NO 5 to SEQ ID NOS 6-10.

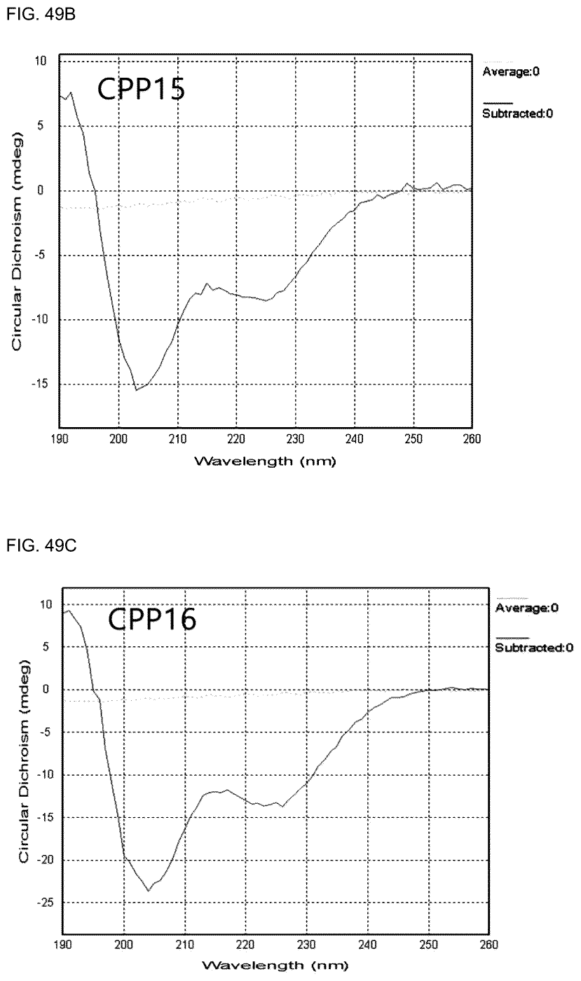

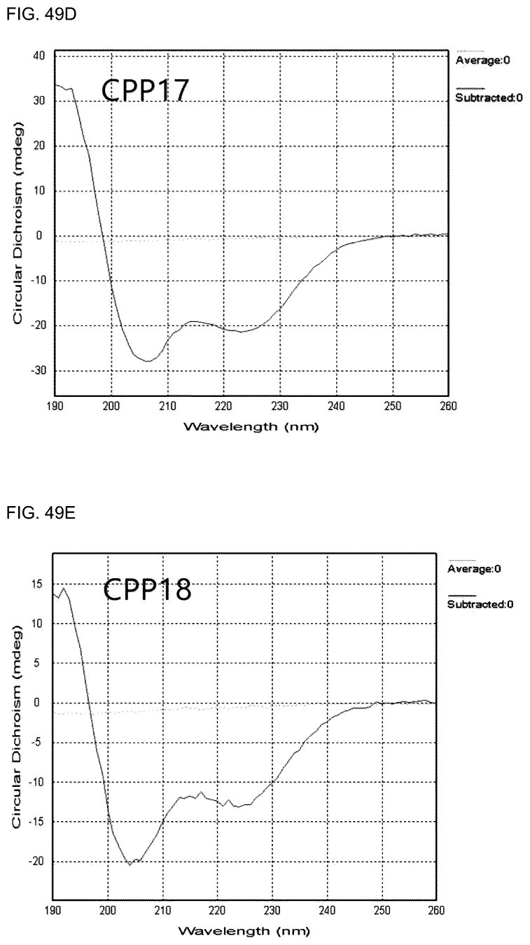

FIGS. 49A-49E show the CD spectra of five cell-penetrating peptides (CPP14 to 18) prepared in Examples 15-19.

As shown in FIGS. 49A-49E, the cell-penetrating peptides of Examples 15-19 had helicity ([θ]₂₂₂/[θ]₂₀₈) of 0.81, 0.66, 0.66, 0.78 and 0.72, respectively. Through this, it was confirmed that the 5-mer repeating unit sequences can be used for cell penetration as long as the helicity is not decreased to 0.6 or lower. But, since the cell-penetrating peptides of the present disclosure showed decreased helicity when the amino acid Ala (A) was located at the 4th position regardless of the positions of Gln (Q) and Asn (N), it was confirmed that it is desirable that the amino acid Ala (A) is located at the 2nd or 3th position in the repeating unit (SEQ ID NO 1) to obtain a cell-penetrating peptide with helicity of 0.7 or higher. In particular, it was confirmed that Gln (Q) is located at the 1st position in the repeating unit (SEQ ID NO 1) to obtain a cell-penetrating peptide having a rigid structure with helicity of 0.8 or higher.

### Test Example 20. Analysis of In-Vivo Targeting Characteristics of Cell-Penetrating Peptides of Examples 15-19 (CPP14 to 18)

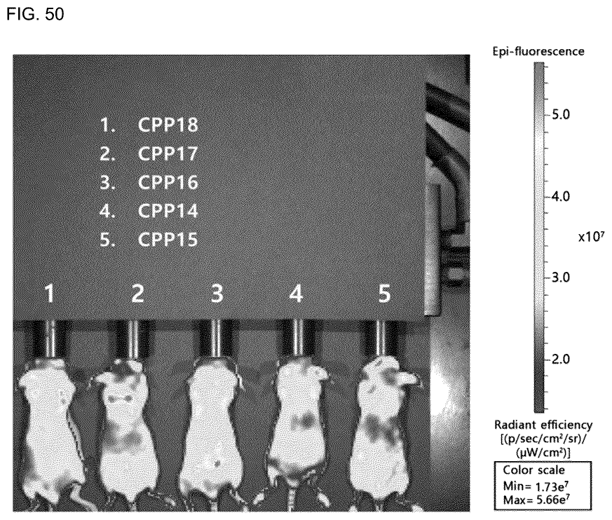

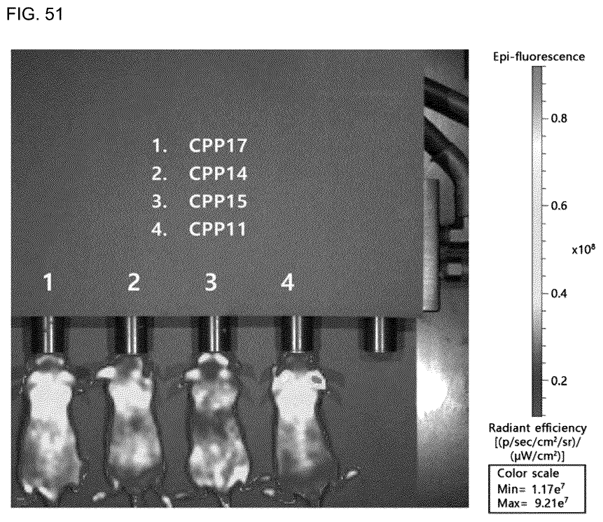

Female 7-week-old BALB/c nude mice were injected IV (intravenously) with 200 μL each of the cell-penetrating peptides of Examples 15-19 (CPP14 to 18) at 5 mg/kg via the lateral tail vein and were imaged using IVIS (in-vivo imaging system) (PerkinElmer, USA) 1 hour later. The images were analyzed with the Living Image software (PerkinElmer) (FIGS. 50 and 51).

Among the cell-penetrating peptides, those exhibiting high accumulation rate were selected and 200 μL was administered IV (intravenously) at 5 mg/kg in the same manner. The cell-penetrating peptide prepared in Example 11 (CPP11) was also administered at the same concentration and then analyzed by IVIS 1 hour later (FIG. 51). The cell-penetrating peptide was used after labeling with FAM.

FIG. 50 shows the IVIS image of the living experimental animals after the administration of the cell-penetrating peptides of Examples 15-19 (CPP14 to 18) by IV injection. FIG. 51 shows the IVIS image of the living experimental animals after the administration of the cell-penetrating peptide of Example 11 (CPP11) and the cell-penetrating peptides of Example 15, 16 and 18 (CPP14, 15 and 17) by IV injection.

As shown in FIG. 50 and FIG. 51, it was confirmed that all of the cell-penetrating peptides of Examples 15-19 (CPP14 to 18) of the same volume target the ear. Among them, the CPP11 prepared in Example 11 showed much higher accumulation efficiency in the ear as compared to other cell-penetrating peptides.

The cell-penetrating peptides according to the present disclosure, which contain the 5-mer repeating unit (SEQ ID NO 1) having an α-helical structure, exhibit remarkable effect in that they target the ear unlike the existing cell-penetrating peptides.

### Test Example 21. Analysis of α-Helix Structure of Cell-Penetrating Peptides (CPP19 to 21)

The structure of the cell-penetrating peptides prepared in Examples 20-22 (CPP19 to CPP21) was analyzed to investigate whether they can easily penetrate cells. The cell-penetrating peptides of Examples 20-22 are cell-penetrating peptide variants wherein A (Arg) in the linker sequence is changed to G (Gly), without change in the 5-mer repeating unit sequence (SEQ ID NO 5).

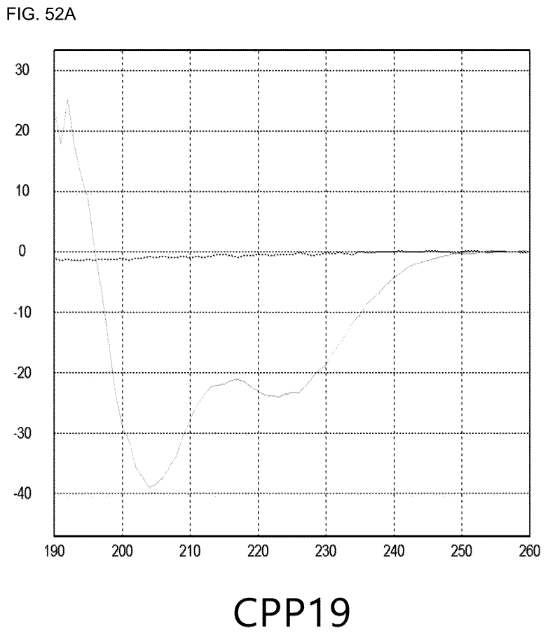

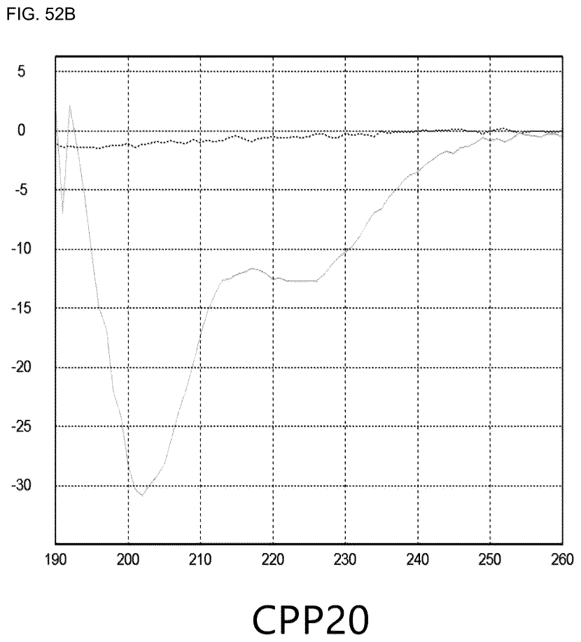

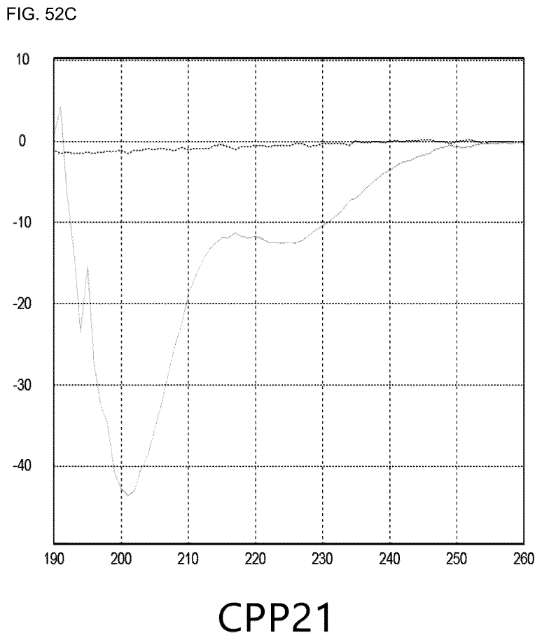

FIGS. 52A-52C show the CD spectra of the cell-penetrating peptides prepared in Examples 20-22 (CPP19 to 21) at 20° C.

As shown in FIG. 52, it was confirmed that the cell-penetrating peptides of Examples 20-22 have helicity ([θ]₂₂₂/[θ]₂₀₈) of 0.706, 0.565 and 0.500, respectively. Through this, it was confirmed that helicity is changed slightly by the substitution of an amino acid of the linker even when the 5-mer repeating unit sequence is identical.

Specifically, when one amino acid was substituted (CPP19, A→G), a cell-penetrating peptide with a helicity of 0.7 or higher could be obtained. But, when one or more amino acid was substituted (CPP20 and 21), the helicity was decreased to 0.5 or lower.

From the experiments described above, it can be seen that the amino acid Ala (A) in the sequence of the linker of the cell-penetrating peptide can be substituted with Gly (G), etc. and, even when 1-3 amino acids are substituted, cell penetration effect can be achieved because the α-helix structure of the cell-penetrating peptide is not completely unfolded. For maintenance of the rigid helical structure and excellent in-vivo delivery, specifically 1-2 amino acids may be substituted, more specifically one amino acid may be substituted, and most specifically no amino acid may be substituted.

### Test Example 22. Analysis of In-Vivo Targeting Characteristics of Cell-Penetrating Peptides of Examples 20-22 (CPP19 to 21) and Cell-Penetrating Peptide of Example 11 (CPP11)

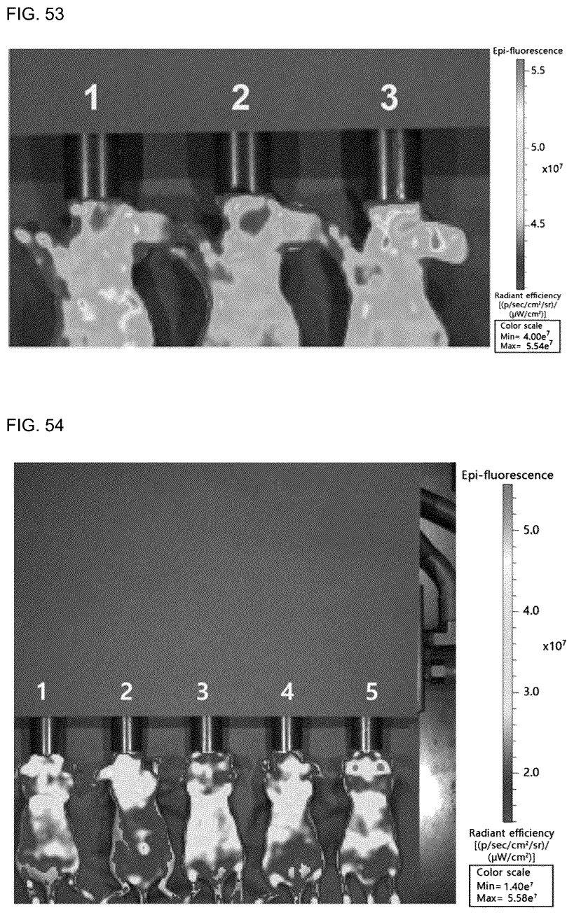

Female 7-week-old BALB/c nude mice were injected IV (intravenously) with 200 μL of the cell-penetrating peptides of Examples 20-22 (CPP19 to 21) at 5 mg/kg via the lateral tail vein. 4 hours later, the mice were imaged using IVIS (in-vivo imaging system) (PerkinElmer, USA) and the images were analyzed with the Living Image software (PerkinElmer) (FIG. 53).

The cell-penetrating peptide prepared in Example 11 (CPP11) was also administered at the same concentration and then analyzed by IVIS 1 hour later (FIG. 53). The cell-penetrating peptide was used after labeling with FAM.

FIG. 53 shows the IVIS image of living experimental animals after administration of the cell-penetrating peptides of Examples 20-22 (CPP19 to 21) (‘3’, ‘2’ and ‘1’ in order) by IV injection.

As shown in FIG. 53, it was confirmed that all of the cell-penetrating peptides of Examples 20, 21 and 22 (CPP19, 21, 22) target the ear. Among them, the CPP20 prepared in Example 19 showed much superior accumulation efficiency in the ear as compared to other cell-penetrating peptides.

The cell-penetrating peptides according to the present disclosure, which contain the 5-mer repeating unit (SEQ ID NO 1) having an α-helical structure, exhibit remarkable effect in that they target the ear unlike the existing cell-penetrating peptides.

### Test Example 23. Analysis of In-Vivo Targeting Characteristics of Cell-Penetrating Peptide of Example 11 (CPP11) and Existing Cell-Penetrating Peptides (Rev ARM, Tat, Penetratin and R8)

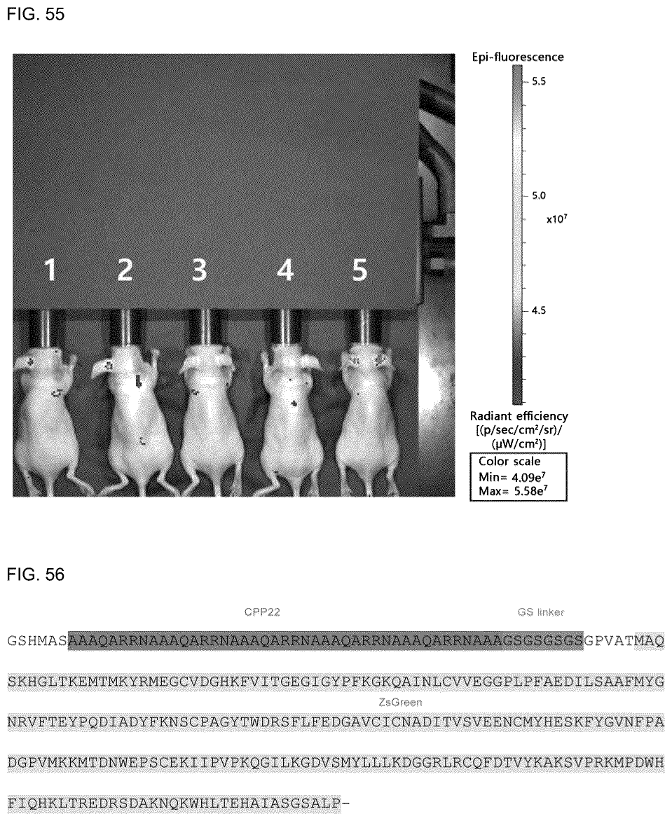

Female 7-week-old BALB/c nude mice were injected IV (intravenously) with 200 μL of the cell-penetrating peptide of Example 11 (CPP11) or the existing cell-penetrating peptides (Rev ARM, Tat, penetratin and R8) at 5 mg/kg via the lateral tail vein. 4 hours later, the mice were imaged using IVIS (in-vivo imaging system) (PerkinElmer, USA) and the images were analyzed with the Living Image software (PerkinElmer) (FIGS. 54 and 55). The cell-penetrating peptides were used after labeling with FAM.

More specifically, the existing cell-penetrating peptides (Rev ARM, Tat, penetratin and R8) were synthesized by standard Fmoc-based solid-phase peptide synthesis using SEQ ID NOS 42, 41, 43 and 44 (see Example 1).

FIG. 54 shows the IVIS image of the living experimental animals after administration of the existing cell-penetrating peptides (Rev ARM, Tat, penetratin and R8) or the cell-penetrating peptide of Example 11 (CPP11) (′1′, ‘2’, ‘3’, ‘4’ and ‘5’ in order) by IV injection. FIG. 55 shows the IVIS image obtained by removing background signals from the IVIS image for the existing cell-penetrating peptides (Rev ARM, Tat, penetratin and R8) or the cell-penetrating peptide of Example 11 (CPP11) (′1′, ‘2’, ‘3’, ‘4’ and ‘5’ in order) (FIG. 54).

As shown in FIG. 54 and FIG. 55, the existing cell-penetrating peptides (Rev ARM, Tat, penetratin and R8) did not show targeting activity for specific sites in the body and did not show significant bioaccumulation. In contrast, the CPP11 prepared in Example 11 was accumulated in the ear tissue at a significantly high level.

The cell-penetrating peptide according to the present disclosure, which contains the 5-mer repeating unit (SEQ ID NO 1) with an α-helical structure, can target the ear despite the substitution in the linker sequence, unlike the existing cell-penetrating peptides.

### Test Example 24. Analysis of Cell Penetration and Delivery Characteristics of Fusion Protein (Example 24) of Cell-Penetrating Peptide of Example 23 (CPP22) and ZsGreen Protein

### Synthesis of CPP22-ZsGreen Fusion Protein

A CPP22-ZsGreen fusion protein represented by SEQ ID NO 34 was synthesized using Rink Amide resin according to the standard Fmoc-based solid-phase peptide synthesis of Example 1 and then purified by RP-HPLC (reversed-phase high-performance liquid chromatography). The structure of the CPP22-ZsGreen fusion protein is shown in FIG. 56. It was intended to investigate whether the cell-penetrating peptide of the present disclosure can be delivered into cells when fused with a cargo protein. The ZsGreen protein refers to the green fluorescent protein.

ZsGreen protein (SEQ ID NO 35) was used as a comparative example. The ZsGreen protein was synthesized by expression from bacterial cells.

### Flow Cytometry (FACS)

Hela cells were inoculated onto a 24-well plate (10⁵ cells/well) and cultured in a DMEM medium containing 10% FBS for 24 hours. After washing with PBS, the cells were treated with ZsGreen or the CPP22-ZsGreen fusion protein at 0, 10, 20 or 50 μM for 4 hours. Then, after washing the cells twice with PBS and incubating with trypsin (1 mg/mL) for 15 minutes, the peptides bound to the cell surface were removed. The cells were centrifuged at 1,200 rpm for 5 minutes, washed twice with PBS, and then suspended in a PBS buffer containing 10% non-enzymatic cell dissociation solution. The fluorescence intensity of the internalized peptide was measured using a BD LSR II flow cytometer (BD Biosciences, USA).

### Confocal Microscopic Analysis

A DMEM medium containing 10% FBS was prepared on an 8-well Lab-Tek II chamber slide (Thermo Fisher Scientific, USA). Hela cells were dispensed in each well at a density of 10⁴ cells/well and cultured for 24 hours. The cells were washed twice with PBS and then cultured for 30 minutes after replacing the medium with one containing 50 μM of the ZsGreen or the CPP22-ZsGreen fusion protein. The cells were washed with PBS, treated with DAPI, and then washed with PBS after reaction for 5 minutes (for observation of cell nuclei). Then, the stained cells were observed by confocal microscopy.

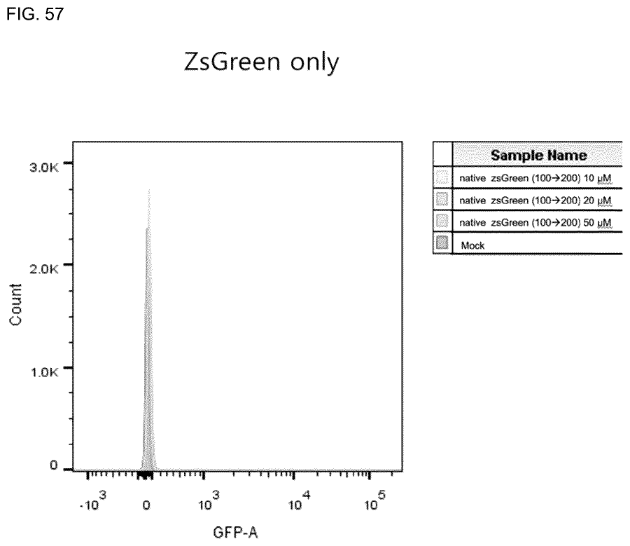

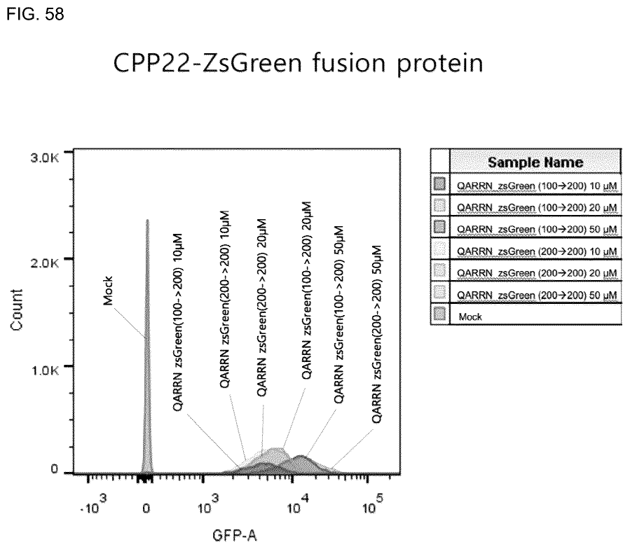

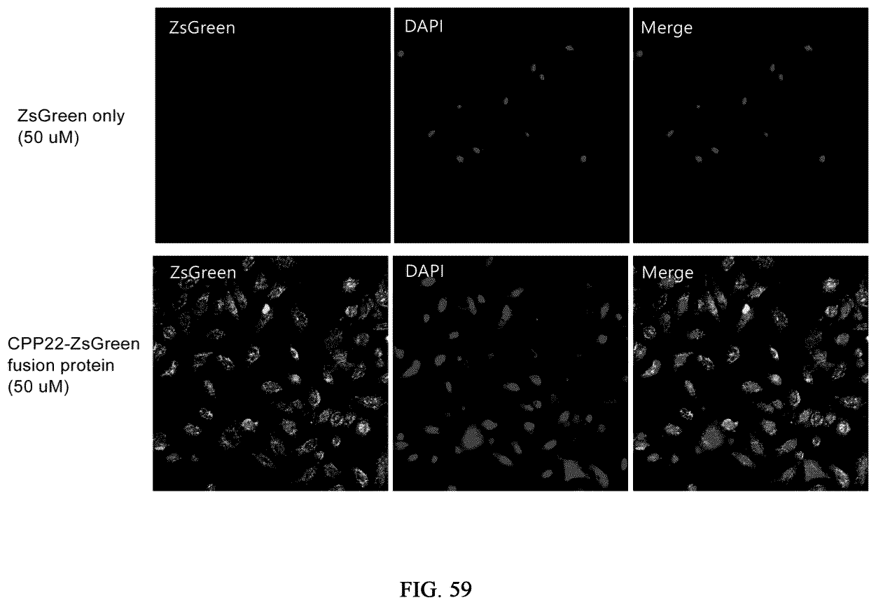

FIG. 57 shows the flow cytometry result for the Hela cells treated only with the ZsGreen protein at various concentrations (10, 20 or 50 μM), and FIG. 58 shows the flow cytometry result for the Hela cells treated with a CPP22-ZsGreen fusion protein at various concentrations (10, 20 or 50 μM).

In the figures, 100→200 and 200→200 refer to the concentration of imidazole used for elution of the proteins in the Ni-NTA column for purification.

As shown in FIGS. 57 and 58, whereas no intracellular delivery was observed in the cells treated only with the ZsGreen protein, the CPP22-ZsGreen fusion protein prepared in Example 24 was delivered to and accumulated in the cells at a significantly high level.

That is to say, it can be seen that the cell-penetrating peptide according to the present disclosure, which contains the 5-mer repeating unit (SEQ ID NO 1) with an α-helical structure, can stably deliver target molecules such as proteins, etc. into cells.

FIG. 59 shows the result of analyzing the Hela cells treated with the ZsGreen protein (50 μM) or the CPP22-ZsGreen fusion protein (50 μM) by confocal microscopy.

As shown in FIG. 59, whereas the ZsGreen protein was not delivered into the cells, the CPP22-ZsGreen fusion protein prepared in Example 23 was successfully delivered into and accumulated in the cells at a significant level.

That is to say, it can be seen that the cell-penetrating peptide according to the present disclosure, which contains the 5-mer repeating unit (SEQ ID NO 1) with an α-helical structure, can stably deliver target molecules such as proteins, etc. into cells.

What is claimed is:

1. A cell-penetrating peptide comprising a repeating unit represented by SEQ ID NO 1:

| [SEQ ID NO 1] | |
|---|---|
| | Xaa1-Xaa2-Xaa3-Xaa4-Xaa5 |

wherein
each of Xaa1 to Xaa5 is independently any one selected from a group consisting of Gln (Q), Ala (A), Arg (R) and Asn (N), the repeating unit being repeated 2-10 times, and
one or more amino acid selected from SEQ ID NO 1 is an L- or D-amino acid.

2. The cell-penetrating peptide according to claim 1, wherein the repeating unit is repeated 2-6 times.

3. The cell-penetrating peptide according to claim 1, wherein the repeating unit is repeated 3-5 times.

4. The cell-penetrating peptide according to claim 1, wherein, in SEQ ID NO 1, each of Xaa1 and Xaa5 is independently Gln (Q) or Asn (N) and each of Xaa2 to Xaa4 is independently Ala (A) or Arg (R).

5. The cell-penetrating peptide according to claim 4, wherein at least one of Xaa2 to Xaa4 is Ala (A).

6. The cell-penetrating peptide according to claim 1, wherein the repeating unit is any one selected from sequences represented by SEQ ID NOS 5-10.

7. The cell-penetrating peptide according to claim 1, wherein the cell-penetrating peptide further comprises a linker represented by any one of SEQ ID NOS 2-4 between the N-terminal and the C-terminal and the repeating unit:

| [SEQ ID NO 2] | |
|---|---|
| | Xaa6 |
| [SEQ ID NO 3] | |
| | Xaa7-Xaa8 |
| [SEQ ID NO 4] | |
| | Xaa9-Xaa10-Xaa11 |

wherein
each of Xaa6 to Xaa11 is independently an α,α-disubstituted amino acid selected from a group consisting of Ala (A), Arg (R), Ile (I), Leu (L), Met (M), Val (V), Gly (G) and 2-aminoisobutyric acid (Aib, U), and
one or more amino acid selected from SEQ ID NOS 2-4 is an L- or D-amino acid.

8. The cell-penetrating peptide according to claim 7, wherein each of Xaa6 to Xaa11 is independently an amino acid selected from Ala (A), Arg (R) and 2-aminoisobutyric acid (Aib, U).

9. The cell-penetrating peptide according to claim 1, wherein the cell-penetrating peptide is any one selected from sequences represented by SEQ ID NOS 11-33.

10. The cell-penetrating peptide according to claim 1, wherein the cell-penetrating peptide is any one selected from sequences represented by SEQ ID NOS 20-33.

11. The cell-penetrating peptide according to claim 1, wherein the cell-penetrating peptide is any one selected from a group consisting of SEQ ID NOS 20, 21, 22, 23, 24 and 33.

12. The cell-penetrating peptide according to claim 1, wherein the cell-penetrating peptide is a cell-penetrating carrier peptide.

13. The cell-penetrating peptide according to claim 1, wherein the cell-penetrating peptide is accumulated in the nucleus of a cell.

14. The cell-penetrating peptide according to claim 1, wherein the cell-penetrating peptide is a cell-penetrating carrier peptide targeting the ear.

15. An intracellular delivery system comprising the cell-penetrating peptide according to claim 1.

16. The intracellular delivery system according to claim 15, wherein a cargo to be delivered intracellularly is bound to the terminal of the cell-penetrating peptide.

17. The intracellular delivery system according to claim 15, wherein the intracellular delivery system targets the ear.

18. A composition for intracellular delivery of an active ingredient, comprising the cell-penetrating peptide according to claim 1 for delivery of an active ingredient into a cell by the cell-penetrating peptide.

19. The composition for intracellular delivery of an active ingredient according to claim 18, wherein the composition targets the ear.

20. The composition for intracellular delivery of an active ingredient according to claim 18, wherein the composition delivers an active ingredient topically into the nucleus of a cell.


SEQUENCE LISTING (44 sequences)

SEQ ID NO: 1-4 - skipped (4 entries)

SEQ ID NO: 5 - protein, 5 aa, synthetic construct
QARRN

SEQ ID NO: 6 - protein, 5 aa, synthetic construct
QRARN

SEQ ID NO: 7 - protein, 5 aa, synthetic construct
QRRAN

SEQ ID NO: 8 - protein, 5 aa, synthetic construct
NRRAQ

SEQ ID NO: 9 - protein, 5 aa, synthetic construct
NRARQ

SEQ ID NO: 10 - protein, 5 aa, synthetic construct
NARRQ

SEQ ID NO: 11 - protein, 24 aa, synthetic construct
RQARRNRQARRNRQARRNRQARRN

SEQ ID NO: 12 - protein, 30 aa, synthetic construct
RQARRNRQARRNRQARRNRQARRNRQARRN

SEQ ID NO: 13 - protein, 36 aa, synthetic construct
RQARRNRQARRNRQARRNRQARRNRQARRNRQARRN

SEQ ID NO: 14 - protein, 28 aa, synthetic construct
AUAUARQARRNRQARRNRQARRNAUAUA

SEQ ID NO: 15 - protein, 39 aa, synthetic construct
UAURQARRNUAURQARRNUAURQARRNUAURQARRNUAU

SEQ ID NO: 16 - protein, 20 aa, synthetic construct
QARRNQARRNQARRNQARRN

SEQ ID NO: 17 - protein, 25 aa, synthetic construct
QARRNQARRNQARRNQARRNQARRN

SEQ ID NO: 18 - protein, 25 aa, synthetic construct
UQARRNUQARRNUQARRNUQARRNU

SEQ ID NO: 19 - protein, 30 aa, synthetic construct
UAQARRNUAQARRNUAQARRNUAQARRNUA

SEQ ID NO: 20 - protein, 27 aa, synthetic construct
UAUQARRNUAUQARRNUAUQARRNUAU

SEQ ID NO: 21 - protein, 35 aa, synthetic construct
UAUQARRNUAUQARRNUAUQARRNUAUQARRNUAU

SEQ ID NO: 22 - protein, 35 aa, synthetic construct
AAAQARRNAAAQARRNAAAQARRNAAAQARRNAAA

SEQ ID NO: 23 - protein, 43 aa, synthetic construct
UAUQARRNUAUQARRNUAUQARRNUAUQARRNUAUQARRNUAU

SEQ ID NO: 24 - protein, 35 aa, synthetic construct; identical to SEQ ID NO: 21
  SITE 2: note = D-Alanine
  SITE 4: note = D-Glutamine
  SITE 5: note = D-Alanine
  SITE 6..7: note = D-Arginine
  SITE 8: note = D-Asparagine
  SITE 10: note = D-Alanine
  SITE 13: note = D-Alanine
  SITE 18: note = D-Alanine
  SITE 21: note = D-Alanine
  SITE 26: note = D-Alanine
  SITE 29: note = D-Alanine
  SITE 34: note = D-Alanine
  SITE 12: note = D-Glutamine
  SITE 20: note = D-Glutamine
  SITE 28: note = D-Glutamine
  SITE 14..15: note = D-Arginine
  SITE 22..23: note = D-Arginine
  SITE 30..31: note = D-Arginine
  SITE 16: note = D-Asparagine
  SITE 24: note = D-Asparagine
  SITE 32: note = D-Asparagine

SEQ ID NO: 25 - protein, 35 aa, synthetic construct
UAUQRARNUAUQRARNUAUQRARNUAUQRARNUAU

SEQ ID NO: 26 - protein, 35 aa, synthetic construct
UAUQRRANUAUQRRANUAUQRRANUAUQRRANUAU

SEQ ID NO: 27 - protein, 35 aa, synthetic construct
UAUNRRAQUAUNRRAQUAUNRRAQUAUNRRAQUAU

SEQ ID NO: 28 - protein, 35 aa, synthetic construct
UAUNRARQUAUNRARQUAUNRARQUAUNRARQUAU

SEQ ID NO: 29 - protein, 35 aa, synthetic construct
UAUNARRQUAUNARRQUAUNARRQUAUNARRQUAU

SEQ ID NO: 30 - protein, 35 aa, synthetic construct
UAUQARRNUAUQARRNUAUQARRNUGUQARRNUAU

SEQ ID NO: 31 - protein, 35 aa, synthetic construct
UAUQARRNUAUQARRNUGUQARRNUGUQARRNUAU

SEQ ID NO: 32 - protein, 35 aa, synthetic construct
UAUQARRNUGUQARRNUGUQARRNUGUQARRNUAU

SEQ ID NO: 33 - protein, 43 aa, synthetic construct
AAAQARRNAAAQARRNAAAQARRNAAAQARRNAAAQARRNAAA

SEQ ID NO: 34 - protein, 293 aa, synthetic construct
GSHMASAAAQARRNAAAQARRNAAAQARRNAAAQARRNAAAQARRNAAAGSGSGSGSGPVATMAQSKHGLTKEMTMKYRMEGCVDGHKFVITGEGIGYPFKGKQAINLCVVEGGPLPFAEDILSAAFMYGNRVFTEYPQDIADYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 35 - protein, 231 aa, synthetic construct
MAQSKHGLTKEMTMKYRMEGCVDGHKFVITGEGIGYPFKGKQAINLCVVEGGPLPFAEDILSAAFMYGNRVFTEYPQDIADYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 36 - protein, 56 aa, synthetic construct
DEDALKAARAIKFLYQSNPPPNPEGTRQARRNRRRRWRERQRQIHSISERILSTLG

SEQ ID NO: 37 - protein, 64 aa, Human immunodeficiency virus type I
CRGGDEDALKAARAIKFLYQSNPPPNPEGTRQARRNRRRRWRERQRQIHSISERILSTLGGMRM

SEQ ID NO: 38 - protein, 64 aa, synthetic construct
  REGION 1..64: note = The peptide is circular.
CRGGDEDALKAARAIKFLYQSNPPPNPEGTRQARRNRRRRWRERQRQIHSASERIASTLGGMRM

SEQ ID NO: 39 - RNA, 46 nt, Human immunodeficiency virus type I
  source 1..46: mol_type = genomic RNA
ggacctggtatgggcgcagcgcaagctgacggtacaggccaggtcc

SEQ ID NO: 40 - RNA, 46 nt, Human immunodeficiency virus type I
  source 1..46: mol_type = genomic RNA
ggacctggtatcggcgcagcgcaagctgacggtagaggccaggtcc

SEQ ID NO: 41 - protein, 11 aa, Human immunodeficiency virus 1
YGRKKRRQRRR

SEQ ID NO: 42 - protein, 17 aa, Human immunodeficiency virus type I
TRQARRNRRRRWRERQR

SEQ ID NO: 43 - protein, 16 aa, synthetic construct
RQIKIWFQNRRMKWKK

SEQ ID NO: 44 - protein, 8 aa, synthetic construct
RRRRRRRR